(19) United States
(12) Patent Application Publication
(10) Pub. No.: US 2025/0352555 A1
(43) Pub. Date: Nov. 20, 2025

(54) METHOD OF INCREASING IMMUNE CELL ACTIVATION AND/OR TREATING CANCER USING DIBENZOXAZEPINONES

(71) Applicant: VASCULAR THERAPEUTICS PTY LTD, Rhodes (AU)

(72) Inventors: Ben Jing Wu, New South Wales (AU); Yue Li, New South Wales (AU); Levon Khachigian, New South Wales (AU)

(21) Appl. No.: 19/120,141

(22) PCT Filed: Oct. 13, 2023
(86) PCT No.: PCT/AU2023/051015

§ 371 Date: Apr. 10, 2025

(30) Foreign Application Priority Data
Oct. 13, 2022 (AU) 2022903003

(51) Int. Cl. A61K 31/553; A61P 35/00; A61P 37/04

(52) U.S. Cl. CPC A61K 31/553; A61P 35/00; A61P 37/04

(57) ABSTRACT

The invention relates to a method of increasing immune cell activation and/or treating cancer in a subject, comprising administering to the subject an effective amount of a compound of formula (I), or a pharmaceutically acceptable salt thereof: H NO R1 ON O R2 O.

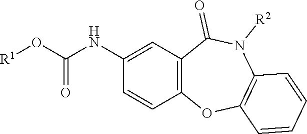

### FIELD OF THE INVENTION

The present invention relates to methods, and pharmaceutical compositions for increasing immune cell activation, and to methods for treating cancer.

### BACKGROUND

Cancer is one of the leading causes of death worldwide. While advances have been made in the developments of therapies for cancer, there remains a substantial need for improved therapies for example, to treat cancers where tumor resistance is a problem for existing therapies, or tumors which are non-responsive to existing therapies. One approach that has shown promise in the treatment of cancer is cancer immunotherapy. Cancer immunotherapy is a form of treatment that involves modulating the patient's immune system to treat the cancer. In cancer immunotherapy, antibodies which target the programmed cell death protein-1 (PD-1) have shown particular promise. PD-1 and its ligand PD-L1 are checkpoint regulators that suppress the body's immune response to cancer cells. PD-L1 is a transmembrane protein expressed by tumor cells, and hematopoietic and non-hematopoietic cells. PD-1 is a co-inhibitory receptor mainly expressed by T cells, and also expressed by B cells, NK cells and certain myeloid cells. PD-1, which is encoded by the pdcd1 gene, binds PD-L1 on the tumor surface and prevents cytolysis by immune cells. PD-1 has been shown to also be expressed by tumor cells including human melanoma cell lines (Kleffel et al., 2015; Li et al., 2019).

A number of immune checkpoint inhibitors against PD-1 and PD-L1 have been developed and have shown promise in treating multiple malignancies. For example, antibodies that have been approved and/or are in clinical trials, for use in treatment of various cancers, include the anti-PD-1 antibodies nivolumab (for metastatic melanoma), pembrolizumab (for treatment of metastatic melanoma, lymphoma, mesothelioma, and non-small cell lung cancer), anti-PD-L1 antibodies including avelumab (for urothelial carcinoma, Merkel cell carcinoma, renal cell carcinoma) and atezolizumab (for urothelial carcinoma, non-small cell lung cancer (NSCLC), triple-negative breast cancer (TNBC), small cell lung cancer (SCLC) and hepatocellular carcinoma (HCC)).

While antibodies against PD-1 have shown promising results in treating cancer, there are limitations to the use of antibody-based therapeutics. For example, 40% of melanoma patients initially responding to a PD-1 inhibitor will progress in existing or new lesions within 3 years. In metastatic melanoma, long-term survival (>3 years) with anti-CTLA-4 is just ˜20% and ˜30-50% when combined with PD-1 antibodies. KEYNOTE-001 revealed that 5 year overall survival for advanced melanoma patients treated with anti-PD-1 (pembrolizumab) was 34% in all patients and 41% in treatment-naive patients, with treatment-related adverse events being recorded in 86% of patients. Combination of anti-PD-1 (nivolumab) and anti-CTLA-4 (ipilimumab) increased the response rate to 53% but severe treatment-related adverse events (grade ≥3) were recorded in 53% of patients and treatment was discontinued in 21%. Of concern are recent studies identifying venous thromboembolism in 24% of cancer patients on immunotherapy with decreased overall survival.

Notwithstanding the need for safer, more effective immune checkpoint therapies, small molecules offer potential advantages over antibodies such as favourable pharmacokinetics and druggability and are amenable to oral formulation and outpatient delivery. This means potential avoidance of intravenous (i.v.) administration and associated risks, and greater patient convenience particularly among the frail. Small molecules are typically cheaper to produce and more stable than antibodies. Despite the PD-1/PD-L1 system now being a staple target in immunotherapy, there are no clinically approved small molecule inhibitors of the PD-1/PD-L1 system.

Inhibitors of BRAF and MEK have also shown promise in the treatment of cancer. For example, first-line treatment of metastatic melanoma patients with dabrafenib and trametinib led to 5-year survival in 1 of 3 melanoma patients with a BRAF^{V600E} or ^{V600K} mutation. However, 30% of patients develop higher grade 3/4 toxicities which often lead to dose reductions and delay of treatment. Importantly, resistance can develop after 9-12 months, likely due to activation of other signalling pathways or modulation of the immune system. Increased downstream ERK signalling is a resistance mechanism to BRAF/MEK inhibition and has led to various pre-clinical and clinical initiatives to target ERK. Moreover, recent studies in non-small cell lung carcinoma-bearing mice show that an ERK inhibitor (PD0325901) can enhance the efficacy of anti-PD-1 antibodies. ASN007 is another ERK1/2 kinase inhibitor which demonstrates efficacy in a resistant melanoma PDX model. However, of the 62 FDA-approved small molecule therapeutic agents targeting over 20 different protein kinases, 8 of which were approved in 2020, none of these are an ERK inhibitor even though several are under clinical investigation (e.g., ulixertinib NCT03698994 National Cancer Institute; LY3214996 NCT02857270, Eli Lilly; BVD-523 NCT03417739; BioMed Valley Discoveries; LTT462 NCT02711345, Novartis; MK-8353 NCT02972034, Merck Sharp & Dohme).

By combining an immune checkpoint inhibitor with targeted therapy, cancer patients may benefit from targeted therapy at least in the short term while immunotherapy may provide a longer-lasting response.

What is needed is a non-antibody alternative for the treatment of cancers which targets the immune checkpoint inhibitor system and ERK signalling.

### SUMMARY

The inventors have found that a compound, BT2 (a compound of formula (II), serves as both an immune checkpoint inhibitor and a targeted inhibitor of ERK signalling. In this regard, the inventors have found that BT2:
(i) inhibits ERK phosphorylation, and increases expression of JUN (also known as c-Jun), in tumor cells; and
(ii) reduces expression of PD-1, increases expression of JUN, increases phosphorylation of JNK, and interacts with CD28, in T cells.

The inventors therefore reasoned that BT2 would be beneficial for increasing immune cell activation, and for the treatment of cancer through its combined activity as an immune checkpoint inhibitor, as an inhibitor of ERK phosphorylation, and a promoter of JUN expression.

A first aspect provides a method of increasing immune cell activation, and/or treating cancer, in a subject, comprising administering an effective amount of a compound of formula (I), or a pharmaceutically acceptable salt thereof:

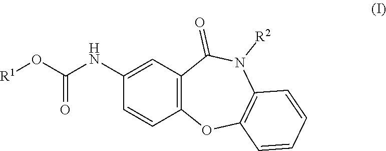

wherein:
R¹ is straight or branched C₁-C₆ alkyl; and
R² is straight or branched C₁-C₆ alkyl,
or R² is

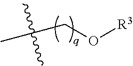

wherein q is 1, 2, 3 or 4; and R³ is straight or branched C₁-C₆ alkyl.

An alternative first aspect provides a compound of formula (I), or a pharmaceutically acceptable salt thereof, for use in increasing immune cell activation, and/or treating cancer, in a subject; or use of a compound of formula (I), or a pharmaceutically acceptable salt thereof, in the manufacture of a medicament for increasing immune cell activation, and/or treating cancer, in a subject.

A second aspect provides a method of increasing immune cell activation, and/or treating cancer, in a subject, comprising administering an effective amount of a compound of formula (II), or a pharmaceutically acceptable salt thereof:

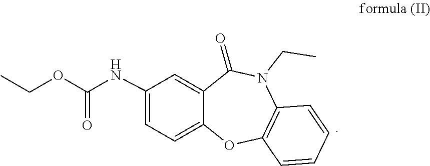 [SMILES: CCOC(=O)Nc1ccc2c(c1)C(=O)N(CC)c1ccccc1O2]

An alternative second aspect provides a compound of formula (II), or a pharmaceutically acceptable salt thereof, for use in increasing immune cell activation, and/or treating cancer, in a subject; or use of a compound of formula (II), or a pharmaceutically acceptable salt thereof, in the manufacture of a medicament for increasing immune cell activation, and/or treating cancer, in a subject.

A third aspect provides a method of reducing ERK phosphorylation and/or increasing JUN expression in a tumor cell of a subject, and/or reducing PD-1 expression and/or increasing JUN expression and/or increasing JNK phosphorylation in T cells of a subject, comprising administering an effective amount of a compound of formula (I), or a pharmaceutically acceptable salt thereof.

An alternative third aspect provides a compound of formula (I), or a pharmaceutically acceptable salt thereof, for use in reducing ERK phosphorylation and/or increasing JUN expression in a tumor cell of a subject, and/or reducing PD-1 expression and/or increasing JUN expression and/or increasing JNK phosphorylation in T cells of a subject; or use of a compound of formula (I), or a pharmaceutically acceptable salt thereof, in the manufacture of a medicament for reducing ERK phosphorylation and/or increasing JUN expression in a tumor cell of a subject, and/or reducing PD-1 expression and/or increasing JUN expression and/or increasing JNK phosphorylation in T cells of a subject.

A fourth aspect provides a method of reducing ERK phosphorylation and/or increasing JUN expression in a tumor cell of a subject, and/or reducing PD-1 expression and/or increasing JUN expression and/or increasing JNK phosphorylation in T cells of a subject, comprising administering an effective amount of a compound of formula (II), or a pharmaceutically acceptable salt thereof.

An alternative fourth aspect provides a compound of formula (II), or a pharmaceutically acceptable salt thereof, for use in reducing ERK phosphorylation and/or increasing JUN expression in a tumor cell of a subject, and/or reducing PD-1 expression and/or increasing JUN expression and/or increasing JNK phosphorylation in T cells of a subject; or use of a compound of formula (II), or a pharmaceutically acceptable salt thereof, in the manufacture of a medicament for reducing ERK phosphorylation and/or increasing JUN expression in a tumor cell of a subject, and/or reducing PD-1 expression and/or increasing JUN expression and/or increasing JNK phosphorylation in T cells of a subject.

A fifth aspect provides a method of reducing ERK phosphorylation in a tumor cell of a subject, and reducing PD-1 expression in T cells of a subject, comprising administering an effective amount of a compound of formula (I), or a pharmaceutically acceptable salt thereof.

An alternative fifth aspect provides a compound of formula (I), or a pharmaceutically acceptable salt thereof, for use in reducing ERK phosphorylation in a tumor cell of a subject, and reducing PD-1 expression in T cells of a subject; or use of a compound of formula (I), or a pharmaceutically acceptable salt thereof, in the manufacture of a medicament for reducing ERK phosphorylation in a tumor cell of a subject, and reducing PD-1 expression in T cells of a subject.

A sixth aspect provides a method of reducing ERK phosphorylation in a cancer cell of a subject, and reducing PD-1 expression in T cells of a subject, comprising administering an effective amount of a compound of formula (II), or a pharmaceutically acceptable salt thereof.

An alternative sixth aspect provides a compound of formula (II), or a pharmaceutically acceptable salt thereof, for use in reducing ERK phosphorylation in a tumor cell of a subject, and reducing PD-1 expression in T cells of a subject; or use of a compound of formula (II), or a pharmaceutically acceptable salt thereof, in the manufacture of a medicament for reducing ERK phosphorylation in a tumor cell of a subject, and reducing PD-1 expression in T cells of a subject.

A seventh aspect provides a method of treating a disease or condition associated with PD-1 expression in T cells of a subject, comprising administering an effective amount of a compound of formula (I), or a pharmaceutically acceptable salt thereof.

An alternative seventh aspect provides a compound of formula (I), or a pharmaceutically acceptable salt thereof, for use in treating a disease or condition associated with PD-1 expression in T cells of a subject; or use of a compound of formula (I), or a pharmaceutically acceptable salt thereof, in the manufacture of a medicament for treating a disease or condition associated with PD-1 expression in T cells of a subject.

An eighth aspect provides a method of treating a disease or condition associated with PD-1 expression in T cells of a subject, comprising administering an effective amount of a compound of formula (II), or a pharmaceutically acceptable salt thereof.

An alternative eighth aspect provides a compound of formula (II), or a pharmaceutically acceptable salt thereof, for use in treating a disease or condition associated with PD-1 expression in T cells of a subject; or use of a compound of formula (II), or a pharmaceutically acceptable salt thereof, in the manufacture of a medicament for treating a disease or condition associated with PD-1 expression in T cells of a subject.

A ninth aspect provides a method of increasing immune cell activation, and/or reducing the rate of tumor growth, in a subject suffering from cancer, comprising administering an effective amount of a compound of formula (I), or a pharmaceutically acceptable salt thereof.

An alternative ninth aspect provides a compound of formula (I), or a pharmaceutically acceptable salt thereof, for use in increasing immune cell activation, and/or reducing the rate of tumor growth, in a subject suffering from cancer; or use of a compound of formula (I), or a pharmaceutically acceptable salt thereof, in the manufacture of a medicament for increasing immune cell activation, and/or reducing the rate of tumor growth, in a subject suffering from cancer.

A tenth aspect provides a method of increasing immune cell activation, and/or reducing the rate of tumor growth, in a subject suffering from cancer, comprising administering an effective amount of a compound of formula (II), or a pharmaceutically acceptable salt thereof.

An alternative tenth aspect provides a compound of formula (II), or a pharmaceutically acceptable salt thereof, for use in increasing immune cell activation, and/or reducing the rate of tumor growth, in a subject suffering from cancer; or use of a compound of formula (II), or a pharmaceutically acceptable salt thereof, in the manufacture of a medicament for increasing immune cell activation, and/or reducing the rate of tumor growth, in a subject suffering from cancer.

An eleventh aspect provides a method of reducing PD-1 expression in T cells of a subject, comprising administering an effective amount of a compound of formula (I), or a pharmaceutically acceptable salt thereof.

An alternative eleventh aspect provides a compound of formula (I), or a pharmaceutically acceptable salt thereof, for use in reducing PD-1 expression in T cells of a subject; or use of a compound of formula (I), or a pharmaceutically acceptable salt thereof, in the manufacture of a medicament for reducing PD-1 expression in T cells of a subject.

A twelfth aspect provides a method of reducing PD-1 expression in T cells of a subject, comprising administering an effective amount of a compound of formula (II), or a pharmaceutically acceptable salt thereof.

An alternative twelfth aspect provides a compound of formula (II), or a pharmaceutically acceptable salt thereof, for use in reducing PD-1 expression in T cells of a subject; or use of a compound of formula (II), or a pharmaceutically acceptable salt thereof, in the manufacture of a medicament for reducing PD-1 expression in T cells of a subject.

A thirteenth aspect provides a method of reducing ERK phosphorylation and/or increasing JUN expression in a tumor cell, and/or reducing PD-1 expression and/or increasing JUN expression and/or increasing JNK phosphorylation in a T cell, comprising contacting the tumor cell or T cell with an effective amount of a compound of formula (I), or a pharmaceutically acceptable salt thereof.

An alternative thirteenth aspect provides a compound of formula (I), or a pharmaceutically acceptable salt thereof, for use in reducing ERK phosphorylation and/or increasing JUN expression in a tumor cell, and/or reducing PD-1 expression and/or increasing JUN expression and/or increasing JNK phosphorylation in a T cell; or use of a compound of formula (I), or a pharmaceutically acceptable salt thereof, in the manufacture of a medicament for reducing ERK phosphorylation and/or increasing JUN expression in a tumor cell, and/or reducing PD-1 expression and/or increasing JUN expression and/or increasing JNK phosphorylation in a T cell.

A fourteenth aspect provides a method of reducing ERK phosphorylation and/or increasing JUN expression in a tumor cell, and/or reducing PD-1 expression and/or increasing JUN expression and/or increasing JNK phosphorylation in a T cell, comprising contacting the tumor cell or T cell with an effective amount of a compound of formula (II), or a pharmaceutically acceptable salt thereof.

An alternative fourteenth aspect provides a compound of formula (II), or a pharmaceutically acceptable salt thereof, for use in reducing ERK phosphorylation and/or increasing JUN expression in a tumor cell, and/or reducing PD-1 expression and/or increasing JUN expression and/or increasing JNK phosphorylation in a T cell; or use of a compound of formula (II), or a pharmaceutically acceptable salt thereof, in the manufacture of a medicament for reducing ERK phosphorylation and/or increasing JUN expression in a tumor cell, and/or reducing PD-1 expression and/or increasing JUN expression and/or increasing JNK phosphorylation in a T cell.

A fifteenth aspect provides a method of reducing expression of a gene listed in Table 2, and/or increasing expression of a gene listed in Table 3, in a T cell, comprising contacting the T cell with an effective amount of a compound of formula (I), or a pharmaceutically acceptable salt thereof.

An alternative fifteenth aspect provides a compound of formula (I), or a pharmaceutically acceptable salt thereof, for use in reducing expression of a gene listed in Table 3, and/or increasing expression of a gene listed in Table 3, in a T cell; or use of a compound of formula (I), or a pharmaceutically acceptable salt thereof, in the manufacture of a medicament for reducing expression of a gene listed in Table 2, and/or increasing expression of a gene listed in Table 3, in a T cell.

A sixteenth aspect provides a method of reducing expression of a gene listed in Table 2, and/or increasing expression of a gene listed in Table 3, in a T cell, comprising contacting the T cell with an effective amount of a compound of formula (II), or a pharmaceutically acceptable salt thereof.

An alternative sixteenth aspect provides a compound of formula (II), or a pharmaceutically acceptable salt thereof, for use in reducing expression of a gene listed in Table 2, and/or increasing expression of a gene listed in Table 3, in a T cell; or use of a compound of formula (II), or a pharmaceutically acceptable salt thereof, in the manufacture of a medicament for reducing expression of a gene listed in Table 2, and/or increasing expression of a gene listed in Table 3, in a T cell.

A seventeenth aspect provides a kit for reducing ERK phosphorylation and/or increasing JUN expression in a tumor cell, and/or reducing PD-1 expression and/or increasing JUN expression and/or increasing JNK phosphorylation in a T cell, the kit comprising a compound of formula (I), or a pharmaceutically acceptable salt thereof.

An eighteenth aspect provides a kit for reducing ERK phosphorylation and/or increasing JUN expression in a tumor cell, and/or reducing PD-1 expression and/or increasing JUN expression and/or increasing JNK phosphorylation in a T cell, the kit comprising a compound of formula (II), or a pharmaceutically acceptable salt thereof.

A nineteenth aspect provides a kit for reducing inflammation in a tumor, and/or reducing PD-1 expression and/or increasing JUN expression and/or increasing JNK phosphorylation in a T cell and/or increasing JUN expression in a tumor cell of a subject, the kit comprising a compound of formula (II), or a pharmaceutically acceptable salt thereof.

### BRIEF DESCRIPTION OF THE FIGURES

Preferred embodiments of the present invention will now be described, by way of example only, with reference to the accompanying drawings:

FIG. 1 shows (A, C) images of Western blots performed with extracts from A375 (A, B) and MeWo (C, D) melanoma cells incubated with 10 or 100 nM of either BT2 (Table 1) or SCH772984 for 24 h and incubated with antibodies to p-ERK or total ERK followed by secondary antibodies. Approximate positions of molecular weight markers are shown. SCH denotes SCH772984. Graphs in (B, D) show band intensity of Western blots quantified using Image J. Plotted data represents mean±SEM. Data represents 2 biologically independent experiments.

FIG. 2 is graphs showing quantification of (A, C) migration into the denuded zone; and (B, D) invasion into the denuded zone of Matrigel® of A375 cells (A, B) and MeWo cells (C, D) after 24 h and 48 h, respectively, following incubation with 1 μM of BT2 or SCH772984. Plotted data represents mean±SEM of the means of 3-4 biologically independent experiments. Statistical significance was assessed by one-way ANOVA or Kruskal Wallis test, as appropriate.

FIG. 3 is images of the morphology of A375 and MeWo cells exposed to 1 μM of vehicle (Veh), BT2 or SCH772984 for 24 h.

FIG. 4 is a graph showing the results of apoptosis assays performed using (A) A375 cells and (B) MeWo cells, exposed to 1 μM of either BT2 or SCH772984 for 24 h. Plotted data (Annexin V-FITC⁺ PI⁻) represents mean±SEM of the means of 3 biologically independent experiments. Statistical significance was assessed by one-way ANOVA.

FIG. 5 shows the results of treatment of C.B.17 SCID mice bearing s.c. melanoma (MDA-MB-435) with BT2 at 20 mg/kg, i.t. or vehicle once a day on a 5 days-on/2 days-off schedule. Treatment commenced on Day 16. (A) is a schematic showing the treatment regimen. (B) is a graph showing tumor growth (volume) and (C) is a graph showing body weight data sourced from the animal study described in Li et al (Li et al., 2019) with the same vehicle group. Data represent mean±SEM. n=10 mice/group. Statistical significance was assessed by t test.

FIG. 6 are graphs showing quantification of immunohistochemical analysis performed with MDA-MB-435 tumors from BT2- or vehicle-treated mice with antibodies to (A, B) p-ERK or (C, D) total ERK. IOD and tissue area were quantified using Image-Pro Plus. Data represent mean±SEM of n=9-10 mice per group. Statistical significance was assessed by Mann-Whitney test.

FIG. 7 shows the effect of treatment of C.B.17 SCID mice bearing s.c. MDA-MB-435 tumors with BT2 200 mg/kg or vehicle i.p. once a day on a 5 days-on/2 days-off schedule on tumor volume and body weight. Treatment commenced on Day 16. (A) is a schematic of the treatment regimen. (B) is a graph showing tumor growth in BT2 and vehicle treated animals and (C) is a graph showing body weight in vehicle and BT2 treated animals. Body weight data is sourced from the animal study described in Li et al (Li et al., 2019) with the same vehicle group. Data represent mean±SEM. n=10 mice/group.

FIG. 8 shows the effect of treatment of C57BL/6J mice bearing s.c. melanoma (B16F10) with an anti-mouse PD-1 monoclonal antibody or control IgG (100 μg, i.p.) twice a week on tumor volume and body weight. Treatment commenced on Day 7. (A) is a schematic of the treatment regimen. (B) is a graph showing change in tumor volume over time for animals treated with BT2 or vehicle. (C) shows measurements of body weight over time. Data represent mean±SEM. n=6 mice/group. Statistical significance was assessed by Mann-Whitney or t test, as appropriate.

FIG. 9 shows the effects of treatment of C57BL/6J mice bearing s.c. B16F10 tumors with BT2 or vehicle (200 mg/kg or 20 ml/kg, respectively, i.p.) once a day on a 5 days-on/2 days-off schedule on tumor volume and body weight. Treatment commenced on Day 5. (A) is a schematic of the treatment regimen. (B) is a graph showing change in tumor volume over time for animals treated with BT2 or vehicle. (C) also shows measurements of body weight over time. Data represent mean±SEM. n=8 mice/group. Statistical significance was assessed by Mann-Whitney or t test, as appropriate.

FIG. 10 are graphs showing individual Day 17 (A) tumor size, and (B) isolated tumor weight from mice treated with BT2 or vehicle. Data represent mean±SEM. n=8 mice/group. Statistical significance was assessed by t test.

FIG. 11 is a graph showing Kaplan-Meier analysis of survival performed with a tumor size limit of 500 mm³ as previously described (Haynes et al., 2018), n=8 mice/group. Statistical significance was assessed by log-rank (Mantel-Cox) text and Gehan-Breslow-Wilcoxon test.

FIG. 12 are graphs showing quantification of immunohistochemical analysis performed with B16F10 tumors (Day 17) from BT2- or vehicle-treated mice with antibodies to (A) p-ERK or (B) total ERK. IOD and tissue area were quantified using Image-Pro Plus. Data represent mean±SEM of n=8 mice per group. Statistical significance was assessed by Mann-Whitney test.

FIG. 13 is a graph showing quantitation of immunohistochemical analysis performed on B16F10 tumors (Day 17) from BT2- or vehicle-treated mice with antibodies to CD68. IOD and tissue area were assessed using Image-Pro Plus and the IOD/μm² was determined. Data represent mean±SEM of the mean/animal. n=8 per group. Statistical significance was assessed by t test.

FIG. 14 are graphs showing quantitation of immunohistochemical analysis performed on B16F10 tumors (Day 17) from BT2- or vehicle-treated mice with antibodies to CD3. (A) IOD and tissue area were assessed using Image-Pro Plus and the IOD/μm² determined. (B) CD3 positive cell number and total cell number were quantified using Image-Pro Plus and % CD3⁺ cells determined. Data represent mean±SEM of the mean/animal. n=7-8 per group. Statistical significance was assessed by Mann-Whitney test.

FIG. 15 is a graph showing serum IFN-γ levels in vehicle or BT2 treated mice determined by ELISA. n=8 per group. Statistical significance was assessed by t test. Data represent mean±SEM.

FIG. 16 are graphs showing quantification of immunohistochemical analysis performed on B16F10 tumors (Day 17) from BT2- or vehicle-treated mice with antibodies to (A) PD-1, and (B) PD-L1. IOD and tissue area were assessed using Image-Pro Plus and the IOD/μm² determined. Data represent mean±SEM of the mean/animal. n=7-8 per group. Statistical significance was assessed by Mann-Whitney test.

FIG. 17 are graphs showing the results of flow cytometry performed: (A) with PD-1 antibodies (or IgG) and Jurkat T cells treated with the indicated concentrations of BT2 or vehicle for 48 h; (B) with PD-1 antibodies (or IgG) and Jurkat T cells treated with vehicle or the indicated concentrations of BT3 for 48 h; and (C) with PD-1 antibodies (or IgG) and Jurkat T cells treated with 3 μM BT2 for the indicated times or vehicle. Statistical significance was assessed by one way ANOVA. Data represent mean±SEM of the means of 3 biologically independent experiments.

FIG. 18 shows (A) an image of a Western blot performed with extracts of Jurkat T cells treated with various concentrations of BT2 or SCH772984 for 24 h. Membranes were incubated with PD-1 or ß-actin antibodies followed by secondary antibodies. (B) is a graph showing band intensity from western blots as in (A) quantified using Image J and plotted data represents mean±SEM. Data represents 2 biologically independent experiments. Approximate positions of molecular weight markers are shown.

FIG. 19 shows (A) an image of a Western blot performed with extracts of Jurkat T cells treated with various concentrations of BT2 or PD98059 for 24 h. Membranes were incubated with PD-1 or ß-actin antibodies followed by secondary antibodies. (B) is a graph showing band intensity from Western blots quantified using Image J and plotted data represents mean±SEM. Data represents 2 biologically independent experiments. Approximate positions of molecular weight markers are shown.

FIG. 20 shows (A) an image of a Western blot performed with extracts of Jurkat T cells incubated with BT2 or PD98059 for 24 h. Membranes were incubated with DUSP8 antibodies followed by secondary antibodies. (B) is a graph showing band intensity from Western blots quantified using Image J and plotted data represents mean±SEM. Data represents 2 biologically independent experiments. Approximate positions of molecular weight markers are shown.

FIG. 21 shows (A) an image of a Western blot performed with extracts of Jurkat T cells incubated with BT2 or PD98059 for 24 h. Membranes were incubated with c-MAF antibodies followed by secondary antibodies. (B) is a graph showing band intensity from Western blots quantified using Image J and plotted data represents mean±SEM. Data represents 2 biologically independent experiments. Approximate positions of molecular weight markers are shown.

FIG. 22 shows (A) images of Western blots performed with extracts from Jurkat T cells incubated with BT2 for various times. Membranes were incubated with the indicated antibodies followed by secondary antibodies. (B) are graphs showing band intensity from Western blots quantified using Image J and plotted data represents mean±SEM. Data represents 2 biologically independent experiments. Approximate positions of molecular weight markers are shown.

FIG. 23 shows images of Western blots performed with extracts from (A) A375, (B) MDA-MB-435 and (C) MeWo cells incubated with indicated amounts of BT2 or SCH772984 for 24 h. Membranes were incubated with JUN or ß-actin antibodies followed by secondary antibodies. Below each Western blot is a graph showing Western blot band intensity quantified using Image J and plotted data represents mean±SEM. Data represents 2 biologically independent experiments. Approximate positions of molecular weight markers are shown.

FIG. 24 is a sensorgram showing BT2 binding to CD28 over the concentration range of BT2 0.156-15 μM. Measurements were made on a Biacore T200 at 12° C. in 20 mM HEPES, 150 mM NaCl, 5% DMSO, pH 7.5.

FIG. 25 shows (A) image of a Western blot in which vehicle, BT2 (3 nmol) or a mix of BT2 (3 nmol) and CD28 (3 nmol) or EGF (3 nmol) was preincubated in growth medium for 30 min at 37° C., then added to Jurkat T cells in 12-well plates (final concentration of BT2, CD28 or EGF was 3 μM). After 24 h, total cell lysates were prepared in RIPA buffer. Membranes were incubated with the indicated primary antibodies followed by secondary antibodies. (B) is a graph showing band intensity from Western blots quantified using Image J and plotted data represents mean±SEM. Data represents 2 biologically independent experiments. Approximate positions of molecular weight markers are shown.

### DETAILED DESCRIPTION

The compound BT2 is a dibenzoxapinone shown previously to inhibit endothelial cell proliferation and migration, angiogenesis and wound repair. BT2 was shown to inhibit ERK phosphorylation and expression of FosB/ΔFosB and VCAM-1, and VEGF, among others, in endothelial cells.

As described in the Examples, the inventors have now found that BT2 (compound of formula (II)):
(a) reduces ERK phosphorylation in cancer cells;
(b) reduces tumor cell migration;
(c) reduces tumor cell invasion;
(d) increases apoptosis in cancer cells;
(e) reduces rate of tumor growth;
(f) reduces tumor volume;
(g) engages CD28 of T cells;
(h) reduces PD-1 expression in T cells;
(i) increases JUN expression in T cells;
(j) increases JUN expression in tumor cells;
(k) increases JNK phosphorylation in T cells;
(l) inhibits tumor inflammation;
(m) increases tumor immunity.

The inventors therefore envisage that the compound of formula (I) and (II) will be effective for increasing immune cell activation, reducing tumor inflammation and for treating cancer.

One aspect provides a method of increasing immune cell activation, and/or treating cancer, in a subject, comprising administering an effective amount of a compound of formula (I), or a pharmaceutically acceptable salt thereof:

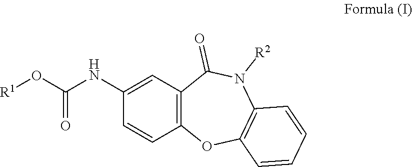

wherein:
R¹ is straight or branched C₁-C₆ alkyl; and
R² is straight or branched C₁-C₆ alkyl,
or R² is

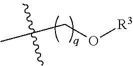

wherein q is 1, 2, 3 or 4; and R³ is straight or branched C₁-C₆ alkyl.

In one embodiment, a compound of formula (I) has the structure of formula (II):

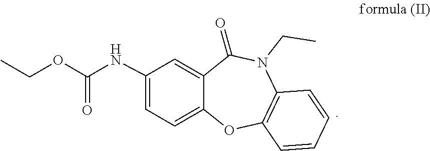 [SMILES: CCOC(=O)Nc1ccc2c(c1)C(=O)N(CC)c1ccccc1O2]

One aspect provides a method of increasing immune cell activation and treating cancer in a subject, comprising administering an effective amount of a compound of formula (I), typically formula (II), or a pharmaceutically acceptable salt thereof.

Another aspect provides a method of reducing ERK phosphorylation in a tumor cell of a subject, and/or reducing PD-1 expression in T cells of a subject, comprising administering an effective amount of a compound of formula (I), or a pharmaceutically acceptable salt thereof:

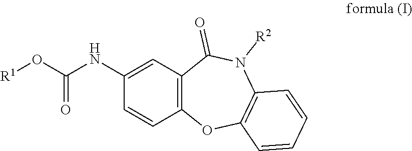

wherein:
R¹ is straight or branched C₁-C₆ alkyl; and
R² is straight or branched C₁-C₆ alkyl,
or R² is

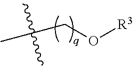

wherein q is 1, 2, 3 or 4; and R³ is straight or branched C₁-C₆ alkyl.

In one embodiment, the compound of formula (I) has the structure of formula (II):

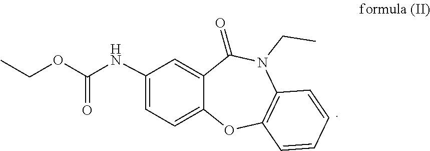 [SMILES: CCOC(=O)Nc1ccc2c(c1)C(=O)N(CC)c1ccccc1O2]

In one embodiment, the method increases JUN expression and JNK phosphorylation in T cells of the subject.

In one embodiment, the method increases JUN expression in tumor cells of the subject.

In one embodiment, the method reduces ERK phosphorylation and increases JUN expression in tumor cells of the subject, and reduces PD-1 expression in T cells of the subject.

In one embodiment, the method reduces ERK phosphorylation and increases JUN expression in tumor cells of the subject, and reduces PD-1 expression in T cells of the subject.

In one embodiment, the method reduces ERK phosphorylation and increases JUN expression in tumor cells of the subject, and increases JUN expression, increases JNK phosphorylation and reduces PD-1 expression in T cells of a subject.

In one embodiment, the method increases DUSP8 expression in T cells of the subject.

In one embodiment, the method reduces MAF expression in T cells of the subject.

In one embodiment, the method reduces tumor cell migration and invasion in the subject.

In one embodiment, the method increases tumor cell apoptosis in the subject.

In one embodiment, the method reduces tumor growth rate in the subject.

In one embodiment, the method reduces tumor cell migration, reduces tumor cell invasion, and increases tumor cell apoptosis, and reduces tumor growth rate, in the subject.

A compound of formula (I) is:

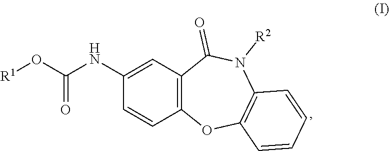

wherein:
R¹ is straight or branched C₁-C₆ alkyl; and
R² is straight or branched C₁-C₆ alkyl,
or R² is

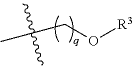

wherein q is 1, 2, 3 or 4; and R³ is straight or branched C₁-C₆ alkyl.

In some embodiments of formula (I), R¹ is straight C₁-C₆ alkyl or branched C₁-C₆ alkyl. In some embodiments of formula (I), R¹ is —CH₂CH₃ or —CH₂CH(CH₃)₂.

In some embodiments of formula (I), R² is straight C₁-C₆ alkyl or branched C₁-C₆ alkyl. In some embodiments of formula (I), R² is —CH₂CH₃ or —CH₂CH(CH₃)₂.

In some embodiments of formula (I), R² is

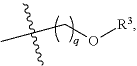

wherein q is 1, 2, 3 or 4; and R³ is straight C₁-C₆ alkyl or branched C₁-C₆ alkyl. In some embodiments of formula (I), q is 2. In some embodiments of formula (I), R³ is —CH₃. In some embodiments of formula (I), q is 2 and R³ is —CH₃.

In some embodiments, the compound of formula (I) may be a compound of formula (I-1):

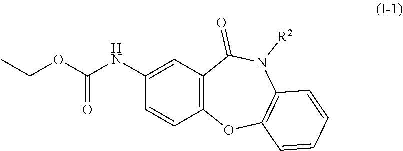

wherein:
R² is straight or branched C₁-C₆ alkyl;
or R² is:

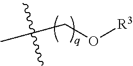

wherein q is 1, 2, 3 or 4; and R³ is straight or branched C₁-C₆ alkyl.

In one embodiment, the compound of formula (I) is the compound of formula (II).

The compound of formula (II) is:

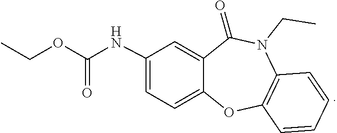 [SMILES: CCOC(=O)Nc1ccc2c(c1)C(=O)N(CC)c1ccccc1O2]

(also referred to herein as BT2)

A compound which increases immune cell activation refers to a compound that induces or causes or promotes immune cells, typically T-cells, to have an increase in biological function or activity following contact with the compound relative to the biological function or activity of the immune cells that have not been in contact with the compound. Examples of increases in immune cell activation include increased T-cell responsiveness to an antigen, increased proliferation, increased secretion of IFN-γ from T-cells, increased expression of JUN in T cells, reduced expression of PD-1 in T cells, increased expression of DUSP8 in T cells, increased phosphorylation of JNK in T cells.

A compound which reduces PD-1 expression is a compound which reduces the amount of PD-1 protein produced by a cell or tissue following contact with the compound or agent relative to the amount of PD-1 protein produced by a cell or tissue which has not been contacted with the compound.

A compound which increases JNK phosphorylation is a compound which increases the extent of JNK phosphorylation in a cell or tissue following contact with the compound relative to the extent of JNK phosphorylation in a cell or tissue that has not been contacted with the compound.

A compound which increases JUN expression is a compound which increases the amount of JUN protein produced by a cell or tissue following contact with the compound relative to the amount of JUN protein produced by a cell or tissue which has not been contacted with the compound.

A compound which reduces ERK phosphorylation is a compound which reduces the extent of ERK phosphorylation in a cell or tissue following contact with the compound relative to the extent of ERK phosphorylation in a cell or tissue that has not been contacted with the compound.

A compound which increases DUSP8 expression is a compound which increases the amount of DUSP8 protein produced by a cell or tissue following contact with the compound relative to the amount of DUSP8 protein produced by a cell or tissue which has not been contacted with the compound.

A compound which reduces MAF expression is a compound which reduces the amount of MAF protein produced by a cell or tissue following contact with the compound or agent relative to the amount of MAF protein produced by a cell or tissue which has not been contacted with the compound.

In one embodiment, the compound increases JUN expression in T cells.

In one embodiment, the compound increases JUN expression in tumor cells.

In one embodiment, the compound reduces PD-1 expression in T cells.

In one embodiment, the compound increases DUSP8 expression in T cells.

In one embodiment, the compound reduces MAF expression in T cells.

In one embodiment, the compound increases circulating IFN-γ.

In one embodiment, the compound increases PD-L1 expression in tumor cells.

In one embodiment, the compound increases netrin-1 expression in T cells.

In one embodiment, the compound engages CD28.

In one embodiment, the compound reduces tumor inflammation and/or tumor size, and/or increases tumor immunity.

In some embodiments, the compound is a pharmaceutically acceptable salt of the compound of formula (I) or formula (II). Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulphuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, trihaloacetic (e.g., trifluoroacetic), methanesulphonic, trihalomethanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

In one embodiment, the compound of formula (I) or (II), or a pharmaceutically acceptable salt thereof, is deuterated.

In one embodiment, the compound of formula (I) or (II), or a pharmaceutically acceptable salt thereof, is an E isomer.

In one embodiment, the compound of formula (I) or (II), or a pharmaceutically acceptable salt thereof, is a Z isomer.

In one embodiment, the compound of formula (I) or (II), or a pharmaceutically acceptable salt thereof, is a mixture of an E isomer and a Z isomer.

Described herein is a pharmaceutical composition comprising a compound of formula (I) or (II), or a pharmaceutically acceptable salt thereof.

The pharmaceutical composition of the present invention may be used in the methods of the invention described herein.

The pharmaceutical composition typically comprises a pharmaceutically acceptable carrier.

The compounds of formula (I) or (II), or a pharmaceutically acceptable salt thereof, may be used to treat any diseases or conditions associated with PD-1 expression in T cells, or which are associated with ERK phosphorylation. A disease or condition is associated with a protein or phosphoprotein if activity of that protein or phosphoprotein is required for development of, and/or maintaining, the disease or condition.

In one embodiment, the disease or condition is cancer. In one embodiment, the compound of formula (I) or (II), or a pharmaceutically acceptable salt thereof, may be used to treat any cancer in which there is PD-1/PD-L1 inhibition of an anti-tumor immune response.

Examples of cancers that may be treated with the compound of formula (I) or (II), or a pharmaceutically acceptable salt thereof, include melanoma, squamous cell carcinoma, basal cell carcinoma, cutaneous sarcoma, Merkel cell carcinoma, head and neck cancer, non-small cell lung cancer, urothelial cancer, thyroid cancer, renal cell carcinoma, breast cancer, and hepatocellular carcinoma.

In one embodiment, the cancer is a cancer in a subject in which T cells of the subject express PD-1.

In one embodiment, the cancer is a cancer in which one or more tumors of the cancer comprise T cells expressing PD-1.

The cancer for treatment with the method described herein may comprise T cells which express PD-1. However, as described in the Examples, the compound described herein does not only inhibit PD-1 expression, but also engages CD28 on T cells and stimulate T cells activity even in tumors that are not responsive to anti-PD-1 antibody.

Therefore, in some embodiments, the cancer may be a cancer that is resistant to treatment with anti-PD-1 antibody therapy.

As described in the Examples, the inventors have further found that the compound of formula (II) is effective against BRAF mutant cells lines and tumors formed from a BRAF mutant cell line. Advantageously, the compound of formula (II) therefore appears capable of treating cancer irrespective of BRAF mutation status of the tumor.

In some embodiments, the cancer comprises cells that are resistant to treatment with dabrafenib and trametinib.

In some embodiments, the cancer comprises BRAF mutant cells.

The methods described herein may involve the administration of a pharmaceutical composition comprising a compound described herein or a pharmaceutically acceptable salt thereof, and a pharmaceutically acceptable carrier.

Described herein is a pharmaceutical composition comprising a compound of formula (I) or (II), or a pharmaceutically acceptable salt thereof, and a pharmaceutically acceptable carrier.

In some embodiments, the carrier is a non-naturally occurring carrier.

In some embodiments, the compound of formula (I) or (II), or a pharmaceutically acceptable salt thereof, may be used in combination with one or more other agents.

It will be understood that the combined administration of a compound of formula (I) or (II), or a pharmaceutically acceptable salt thereof, with the one or more other agents may be concurrent, sequential or separate administration.

The term “composition” encompasses formulations comprising the active ingredient with conventional carriers and excipients, and also formulations with encapsulating materials as a carrier to provide a capsule in which the active ingredient (with or without other carriers) is surrounded by the encapsulation carrier. In pharmaceutical compositions, the carrier is “pharmaceutically acceptable” meaning that it is compatible with the other ingredients of the composition and is not deleterious to a subject. The pharmaceutical compositions of the present invention may contain other agents or further active agents as described above, and may be formulated, for example, by employing conventional solid or liquid vehicles or diluents, as well as pharmaceutical additives of a type appropriate to the mode of desired administration (for example, excipients, binders, preservatives, stabilizers, flavours, etc.) according to techniques such as those known in the art of pharmaceutical formulation (see, for example, Remington: The Science and Practice of Pharmacy, 21st Ed., 2005, Lippincott Williams & Wilkins).

The pharmaceutical composition may be suitable for intravitreal, oral, rectal, nasal, topical (including dermal, buccal and sub-lingual), vaginal or parenteral (including intramuscular, sub-cutaneous and intravenous) administration or in a form suitable for administration by inhalation or insufflation.

In some embodiments, the compounds described herein may be formulated for administration in, for example, nanoparticles or liposomes, or polymer formulations. Methods for the production of formulations comprising liposome, lipid nanoparticles and polymeric formulations are known in the art and described in, for example, Neervannan, 2006; Zhang et al., 2022. Liposomes, nanoparticles or polymer formulations may comprise cationic lipids such as DOTAP, DOPE, DC-Chol/DOPE, DOTMA, and DOTMA/DOPE, polymers such as hydroxypropyl methylcellulose (HPMC), polyethylene glycol (PEG), poly(lactic acid-co-glycolic acid (PLGA), poly(lactic acid) (PLA), poly(glycolic acid) (PGA).

The compounds described herein or a pharmaceutically acceptable salt thereof, together with a pharmaceutically acceptable carrier, may thus be placed into the form of pharmaceutical compositions and unit dosages thereof. The pharmaceutical composition may be a solid, such as a tablet or filled capsule, or a liquid such as solution, suspension, emulsion, elixir, or capsule filled with the same, for oral administration. The pharmaceutical composition may be a liquid such as solution, suspension, or emulsion, for intravitreal administration. The pharmaceutical composition may also be in the form of suppositories for rectal administration or in the form of sterile injectable solutions for parenteral (including subcutaneous) use.

Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

For preparing pharmaceutical compositions from the compounds described herein, pharmaceutically acceptable carriers can be either solid or liquid. Solid form preparations include powders, tablets, pills, capsules, cachets, lozenes (solid or chewable), suppositories, and dispensable granules. A solid carrier can be one or more substances which may also act as diluents, flavouring agents, solubilisers, lubricants, suspending agents, binders, preservatives, tablet disintegrating agents, or an encapsulating material.

Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Tablets, powders, capsules, pills, cachets, and lozenges can be used as solid forms suitable for oral administration.

Liquid form preparations include solutions, suspensions, and emulsions, for example, water or water-propylene glycol solutions. For example, parenteral injection liquid preparations can be formulated as solutions in aqueous polyethylene glycol solution.

Sterile liquid form compositions include sterile solutions, suspensions, emulsions, syrups and elixirs. The active ingredient can be dissolved or suspended in a pharmaceutically acceptable carrier, such as sterile water, sterile organic solvent or a mixture of both.

The pharmaceutical compositions according to the present invention may thus be formulated for parenteral administration (e.g., by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilisation from solution, for constitution with a suitable vehicle, e.g., sterile, pyrogen-free water, before use.

Pharmaceutical forms suitable for injectable use include sterile injectable solutions or dispersions, and sterile powders for the extemporaneous preparation of sterile injectable solutions. They should be stable under the conditions of manufacture and storage and may be preserved against oxidation and the contaminating action of microorganisms such as bacteria or fungi.

The solvent or dispersion medium for the injectable solution or dispersion may contain any of the conventional solvent or carrier systems for injectable solutions or dispersions, and may contain, for example, water, ethanol, polyol (for example, glycerol, propylene glycol and liquid polyethylene glycol, and the like), suitable mixtures thereof, and vegetable oils.

Pharmaceutical forms suitable for injectable use may be delivered by any appropriate route including intravenous, intramuscular, intracerebral, intrathecal, epidural injection or infusion.

Sterile injectable solutions are prepared by incorporating the active ingredient in the required amount in the appropriate solvent with various other ingredients such as those enumerated above, as required, followed by filtered sterilization. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile-filtered solution of the active ingredient plus any additional desired ingredients. The formulation may also be sterilized by heat treatment (e.g., boiled) or autoclave.

The compounds described herein may be formulated into compositions suitable for oral administration, for example, with an assimilable edible carrier, or enclosed in hard or soft shell gelatin capsule, or compressed into tablets, or incorporated directly with the food of the diet. For oral therapeutic administration, the active compound may be incorporated with excipients and used in the form of ingestible tablets, buccal tablets, troches, capsules, elixirs, suspensions, syrups, wafers, and the like.

As described in the Examples, compound of Formula (II) when was bioavailable when administered orally and intraperitoneally.

One aspect provides a pharmaceutical composition comprising a compound of Formula (I) or (II), or a pharmaceutically acceptable salt thereof, a surfactant, and a solvent or polymer.

In one embodiment, the surfactant is a polysorbate. In one embodiment, the polysorbate is Tween 80.

In one embodiment the solvent is a polar aprotic solvent. In one embodiment, the polar aprotic solvent is dimethylsulfoxide (DMSO).

In one embodiment, the polymer is hydroxypropyl methylcellulose (HPMC).

The amount of active compound in therapeutically useful compositions should be sufficient that a suitable dosage will be obtained.

The tablets, troches, pills, capsules, lozenges, implants and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active ingredient(s) may be incorporated into sustained-release preparations and formulations, including those that allow specific delivery of the active ingredient to specific regions of the gut.

Aqueous solutions suitable for oral use can be prepared by dissolving the active component in water and adding suitable colorants, flavours, stabilising and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Pharmaceutically acceptable carriers include any and all pharmaceutically acceptable solvents, dispersion media, coatings, antibacterial and antifungal agents, isotonic and absorption delaying agents and the like.

Also included are solid form preparations that are intended to be converted, shortly before use, to liquid form preparations for oral administration. Such liquid forms include solutions, suspensions, and emulsions. These preparations may contain, in addition to the active component, colorants, flavours, stabilisers, buffers, artificial and natural sweeteners, dispersants, thickeners, solubilising agents, and the like.

For topical administration, the compounds described herein may be formulated as an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions for nasal administration may be applied directly to the nasal cavity by conventional means, for example with a dropper, pipette or spray. The formulations may be provided in single or multidose form. In the case of a dropper or pipette, this may be achieved by the patient administering an appropriate, predetermined volume of the solution or suspension. In the case of a spray, this may be achieved for example by means of a metering atomising spray pump. To improve nasal delivery and retention the compounds of the invention may be encapsulated with cyclodextrins, or formulated with other agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas.

The aerosol may conveniently also contain a surfactant such as lecithin. The dose of the active ingredient may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the active ingredient will generally have a small particle size for example of the order of 5 to 10 microns or less. Such a particle size may be obtained by means known in the art, for example by micronisation.

When desired, formulations adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

It is especially advantageous to formulate parenteral compositions in dosage unit form for ease of administration and uniformity of dosage. Parental compositions may be in the form of physically discrete units suited as unitary dosages for the subjects to be treated, each unit containing a predetermined quantity of the active ingredient calculated to produce the desired therapeutic effect in association a pharmaceutical carrier.

The compounds may also be administered in the absence of carrier where the compounds are in unit dosage form.

The term “effective amount” refers to the amount of a compound effective to achieve the desired response.

An effective amount of a compound described herein, or a pharmaceutically acceptable salt thereof, can be determined by a person skilled in the art having regard to the particular compound.

It will be understood that the specific dose level and frequency of dosage for any particular subject may be varied and will depend upon a variety of factors including the activity of the specific compound employed, the metabolic stability and length of action of that compound, the age, body weight, general health, sex and diet of the subject, the mode and time of administration, rate of excretion, drug combinations, and the severity of the particular condition.

Suitable dosages of the compounds described herein or further active agents administered in combination with compounds described herein can be readily determined by a person skilled in the art having regard to the particular compound of the invention or further active agent selected.

It will further be understood that when the compounds described herein are to be administered in combination with one or more agents, or other active agents, the dosage forms and levels may be formulated for either concurrent, sequential or separate administration or a combination thereof.

The methods of the present invention are intended for use with any subject that may experience the benefits of the methods of the invention. Thus, the term “subject” includes humans as well as non-human mammals. The subject may, for example, be a domestic animal, zoo animal or livestock.

Unless otherwise herein defined, the following terms will be understood to have the general meanings which follow. The terms referred to below have the general meanings which follow when the term is used alone and when the term is used in combination with other terms, unless otherwise indicated. Hence, for example, the definition of “alkyl” applies to “alkyl” as well as the “alkyl” portions of “haloalkyl”, “heteroalkyl”, “arylalkyl” etc.

The term “alkyl” refers to a straight chain or branched chain saturated hydrocarbyl group. Unless indicated otherwise, preferred are C₁₋₆alkyl and C₁₋₄alkyl groups. The term “C_{x-y}alkyl”, where x and y are integers, refers to an alkyl group having x to y carbon atoms. For example, the term “C₁₋₆alkyl” refers to an alkyl group having 1 to 6 carbon atoms. Examples of C₁₋₆alkyl include methyl (Me), ethyl (Et), propyl (Pr), isopropyl (i-Pr), butyl (Bu), isobutyl (i-Bu), sec-butyl (s-Bu), tert-butyl (t-Bu), pentyl, neopentyl, hexyl and the like. Unless the context requires otherwise, the term “alkyl” also encompasses alkyl groups containing one less hydrogen atom such that the group is attached via two positions, i.e., divalent.

As used herein, “treating” means affecting a subject, tissue or cell to obtain a desired pharmacological and/or physiological effect and includes inhibiting the condition, i.e., arresting its development; or relieving or ameliorating the effects of the condition i.e., cause reversal or regression of the effects of the condition. As used herein, “preventing” means preventing a condition from occurring in a cell or subject that may be at risk of having the condition, but does not necessarily mean that condition will not eventually develop, or that a subject will not eventually develop a condition. Preventing includes delaying the onset of a condition in a cell or subject.

The term “effective amount” refers to the amount of the compound that will elicit the biological or medical response of a tissue, system, animal or human that is being sought by the researcher, veterinarian, medical doctor or other clinician.

TABLE 1
Compounds

| Com- | | |
|---|---|---|
| pound | | |
| name | Chemical name | Chemical structure |
| BT2 | (10-ethyl-11-oxo-10,11-dihydro- dibenzo[b,f][1,4]oxazepin-2-yl)- carbamic acid ethyl ester (CAS 922029-50-3) | 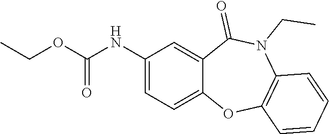 [SMILES: CCOC(=O)Nc1ccc2c(c1)C(=O)N(CC)c1ccccc1O2] |
| BT3 | 2-amino-10- ethyldibenzo[b,f][1,4] oxazepin-11(10H)-one (CAS 23474-61-5) | 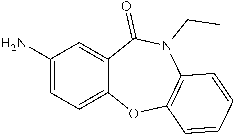 [SMILES: CCN1C(=O)c2cc(N)ccc2Oc2ccccc21] |
| BT2- MeOA | N-(10-ethyl-11-oxo-10,11- dihydrodibenzo[b,f][1,4] oxazepin-2-yl)-2-methoxyacetamide (CAS 922029-59-2) | 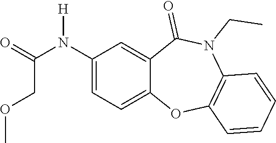 [SMILES: CCN1C(=O)c2cc(NC(=O)COC)ccc2Oc2ccccc21] |
| BT2- EOMe | ethyl (10-(2-methoxyethyl)-11- oxo-10,11- dihydrodibenzo[b,f][1,4] oxazepin-2-yl)carbamate | 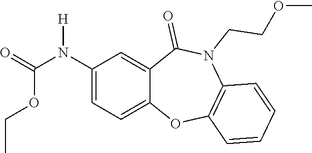 [SMILES: CCOC(=O)Nc1ccc2c(c1)C(=O)N(CCOC)c1ccccc1O2] |
| BT2- Pr | ethyl (11-oxo-10-propyl-10,11- dihydrodibenzo[b,f][1,4] oxazepin-2-yl)carbamate (CAS 922029-50-3) | 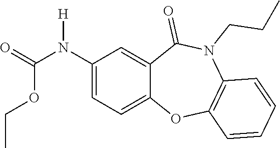 [SMILES: CCCN1C(=O)c2cc(NC(=O)OCC)ccc2Oc2ccccc21] |
| BT2- IC | isobutyl (10-ethyl-11-oxo- 10,11- dihydrodibenzo[b,f][1,4] oxazepin-2-yl)carbamate | 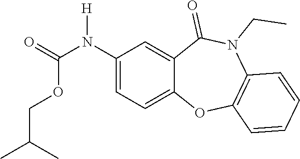 [SMILES: CCN1C(=O)c2cc(NC(=O)OCC(C)C)ccc2Oc2ccccc21] |
| BT2- IMO | ethyl (11-(oxetan-3- ylmethoxy)dibenzo[b,f][1,4] oxazepin-2-yl)carbamate | 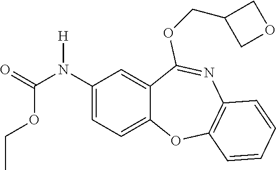 [SMILES: CCOC(=O)Nc1ccc2c(c1)C(OCC1COC1)=Nc1ccccc1O2] |
| BT2- MO | ethyl (10-(oxetan-3-ylmethyl)- 11-oxo-10,11- dihydrodibenzo[b,f][1,4] oxazepin-2-yl)carbamate | 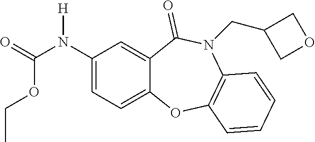 [SMILES: CCOC(=O)Nc1ccc2c(c1)C(=O)N(CC1COC1)c1ccccc1O2] |
| BT2- deut | ethyl (10-ethyl(2′,2′,2′-d3)-11- oxo-10,11- dihydrodibenzo[b,f][1,4] oxazepin-2-yl)carbamate | 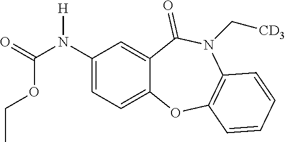 [SMILES: [2H]C([2H])([2H])CN1C(=O)c2cc(NC(=O)OCC)ccc2Oc2ccccc21] |

The compounds described herein may be synthesised by methods known in the art. The compounds referred to herein as BT2 are commercially available. For example, BT2 can be purchased from Aurora Building Blocks, USA, or Life Chemicals HTS Compounds, Canada.

All publications mentioned in this specification are herein incorporated by reference. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present invention is further described below by reference to the following non-limiting Examples.

### EXAMPLES

Here we report that BT2, a dibenzoxazepinone (Li et al., 2020) serves as a novel pharmaco-immunotherapeutic agent and inhibits tumor growth by a dual mechanism. In tumor cells, BT2 serves as an inhibitor of ERK phosphorylation and stimulates JUN expression, while in T cells, BT2 engages CD28, activates JNK phosphorylation, stimulates JUN expression and suppresses PD-1 expression.

### Materials and Methods

Compound synthesis and purification. Compounds BT2, BT3, BT2-MeOA, BT2-EOMe, BT2-Pr, BT2-IC, BT2-IMO, BT2-MO, and BT2-deut were synthesized and purified (>95%) as described in WO 2021/184059.

Cell culture. Human A375 and MeWo melanoma cells were a kind gift of Dr Helen Rizos (Department of Biomedical Sciences, Macquarie University, Sydney). A375 cells were grown in Dulbecco's modified Eagle's medium (DMEM), pH 7.4 containing 10% fetal bovine serum (FBS) in a humidified incubator at 37° C. with 5% CO₂ and routinely passaged following trypsin treatment of adherent cells. MeWo cells were grown in Roswell Park Memorial Institute (RPMI) 1640 medium, pH 7.4 containing 10% FBS. Murine B16F10 and human MDA-MB-435 (American Type Culture Collection, USA) cells were grown in DMEM, pH 7.4 containing 10% FBS. Jurkat T cells (ATCC) were grown in RPMI-1640 medium, pH 7.4 containing 10% FBS.

Western blotting. Cells were treated with compounds and times indicated and total cell lysates were prepared in RIPA buffer. Lysates (10 μg) were resolved by SDS-PAGE and transferred to Immobilon-P polyvinylidene difluoride (PVDF) membranes (Millipore, USA).

Membranes were blocked with 5% skim milk and incubated with rabbit monoclonal anti-c-Jun (Abcam, cat. no. ab32137; mouse, rat and human reactive) antibody, rabbit monoclonal anti-PD-1 (Abcam, cat. no. ab214421) antibody, mouse monoclonal β-actin antibody (Sigma-Aldrich, cat. no. A5316), anti-phospho-SAPK/JNK (Thr¹⁸³/Tyr¹⁸⁵) (98F2) (CST, cat. no. 4671) antibody, anti-phospho-p44/42 MAPK (ERK1/2) (Thr²⁰²/Tyr²⁰⁴) (D13.14.4E) (CST, cat. no. 4370) antibody, anti-phospho-p38 MAPK (Thr¹⁸⁰/Tyr¹⁸²) (D3F9) (CST, cat. no. 4511) antibody, anti-SAPK/JNK antibody (CST, cat. no. 9252), anti-p44/42 MAPK (ERK1/2) (137F5) antibody (CST, cat. no. 4695), anti-p38 MAPK antibody (CST, cat. no. 9212), anti-DUSP8 antibody (Abcam, cat. no. ab198175) or anti-c-MAF (BLR045F) antibody (Abcam, cat. no. ab243901). This was followed by incubation with horseradish peroxidase-conjugated secondary goat anti-rabbit (DAKO, cat. no. P0448) or goat anti-mouse (DAKO, cat. no. P0447) antibody. Chemiluminescence was detected using the Western Lightning Chemiluminescence system (Thermo Scientific, USA) and ImageQuant™ LAS 4000 biomolecular imager (GE Healthcare Life Sciences, USA). Band intensity in images generated with the LAS 4000 was quantified using NIH Image J.

Migration assays. Cells were grown in medium containing 10% FBS in 6-well plates. The following day the cells were scratched with a sterile toothpick, washed with PBS, and incubated in medium containing 10% FBS and vehicle, BT2 or SCH772984 at 1 μM. At 0, 24 h and 48 h the cells were photographed under a 4× objective using an Olympus CKX41 microscope.

Invasion assays. Cells were grown in medium containing 10% FBS in 24-well plates. The following day the cells were scratched with a sterile toothpick, washed with PBS, and overlayed with 90% Matrigel (cat. no. 354230, Corning) (200 μl/well) containing vehicle, BT2 or SCH772984 at 1 μM. The cells were immediately photographed under a 10× objective using an Olympus CKX41 microscope, incubated at 37° C. for 1 h, and medium (800 μl/well) containing 10% FBS with vehicle, BT2 or SCH772984 at 1 μM was added. After 24 or 48 h, the cells were again photographed under a 10× objective using the same microscope.

Cell morphology studies. Cells were grown in medium containing 10% FBS in 4-well chamber slides. The following day the cells were incubated in medium containing 10% FBS and vehicle, BT2 or SCH772984 at 1 μM for 24 h, then fixed with 4% paraformaldehyde solution for 15 min. After washing briefly in PBS the cells were stained with hemotoxylin and eosin. The slides were scanned using an Aperio ScanScope XT slide scanner (Leica Biosystems, Mt Waverley, Vic, Australia) and images were captured using ImageScope software (Leica Biosystems).

Flow cytometry with Annexin V-FITC. Cells were seeded into 6 well plates in normal culture medium containing 10% FBS. After culturing overnight, the cells were incubated in complete medium containing vehicle, BT2 or SCH772984 at 1 μM and left for 24 h. The culture medium was removed and the cells were washed with PBS. Accutase (Stem Cell Technologies, cat. no. 07920) was used to detach the cells. The cells were then washed and centrifuged at 300 g for 5 min and resuspended at 1×10⁶ cells/ml in 500 μl of 1× binding buffer (Annexin V-FITC Apoptosis Staining/Detection Kit, Abcam, cat. no. ab14085). The cells were transferred to 12×75 mm tubes and annexin V-FITC/propidium iodide (PI) was added and incubated for 5 min at 22° C. protected from light. Stained cell suspensions were analyzed by flow cytometry using a BD LSRFortessa X20.

Flow cytometry with anti-PD-1 antibodies. Jurkat T cells were grown in 6 well plates containing RPMI 1640 and 10% FBS. After 24 h, the cells were treated with various concentrations of BT2 or BT3 for 48 h. Alternatively, the cells were incubated with 3 μM BT2 for various times. After treatment, the cells were washed with PBS, and centrifuged at 300×g for 5 min, and resuspended at 5×10⁶ cells/ml. The cells were then incubated with BV421 conjugated mouse anti-human CD279 (PD-1) (BD, cat. no. 562516) or BV421 conjugated mouse IgG1 (BD, cat. no. 562438) for 45 min at 22° C. Cells were then washed twice with 1 ml of Stain Buffer, centrifuged and pellets were resuspended in 0.5 ml Stain Buffer. Stained cell suspensions were analyzed by flow cytometry using a BD FACSCanto (II).

CD28 blockade experiments. BT2 (3 nmol), vehicle or a mix of BT2 (3 nmol) and recombinant CD28 (3 nmol, Sino biological cat. no. 90182-C08H) or EGF (3 nmol, Sigma cat. no. E9644) in 150 μl growth medium (10% FBS/RPMI 1640 medium) was preincubated for 30 min at 37° C. The mixtures were added to 12-well plates containing 850 μl of 10% FBS/RPMI 1640 medium and 0.5×10⁶ Jurkat T cells/well. After 24 h, total cell lysates were prepared in RIPA buffer. Lysates (10 μg) were resolved by SDS-PAGE and transferred to Immobilon-P polyvinylidene difluoride (PVDF) membranes (Millipore, USA). Membranes were blocked with 5% skim milk and incubated with rabbit monoclonal anti-JUN (Abcam, cat. no. ab32137) antibody, mouse monoclonal β-actin antibody (Sigma-Aldrich, cat. no. A5316), This was followed by incubation with horseradish peroxidase-conjugated secondary goat anti-rabbit (DAKO, cat. no. P0448) or goat anti-mouse (DAKO, cat. no. P0447) antibody. Chemiluminescence was detected using the Western Lightning Chemiluminescence system (Thermo Scientific, USA) and ImageQuant™ LAS 4000 biomolecular imager (GE Healthcare Life Sciences, USA). Band intensity in images generated with the LAS 4000 was quantified using NIH Image J.

Melanoma growth studies in mice. Six-to-eight week old C57BL/6J mice (sourced from Australian Resource Centre, Perth) were inoculated subcutaneously (s.c.) with B16F10 cells (1×10⁵ cells/mouse in 100 μl DMEM containing 10% FBS with 50% Matrigel). InVivoPlus anti-mouse PD-1 (CD279) (BioXCell, BP0033-2) or InVivoPlus polyclonal Armenian hamster IgG (Bio X Cell, BP0091) was administered intraperitoneally (i.p.) (100 μg) twice a week. Treatment commenced on Day 7.

Alternatively, BT2 was suspended at 10 mg/ml in vehicle (saline containing 0.5% (v/v) Tween 80 and 0.01% (v/v) DMSO) and sonicated prior to administration. Vehicle or BT2 was administered i.p. (20 ml/kg and 200 mg/kg, respectively) once a day on a 5 days-on/2 days-off schedule. Treatment commenced when tumors were palpable on Day 5.

In experiments with SCID mice, 5 week old CB17/lcr-Prkdcscid/lcrlcoCrl mice (sourced from Charles River Laboratories, USA) were inoculated s.c. with human MDA-MB-435 cells (2.5×10⁶ cells/animal in 100 μl PBS) in the third mammary fat pad on Day 0. These cells were also sourced from ATCC. BT2 was suspended at 9.6 mg/ml in vehicle (saline containing 0.5% (v/v) Tween 80 and 0.01% (v/v) DMSO) and sonicated. Vehicle or BT2 were administered i.p. or intratumorally (i.t.) 20 mg/kg (i.t.) or 200 mg/kg (i.p.), once a day on a 5 days-on/2 days-off schedule. Treatment commenced on Day 16 and ended on Day 43. Tumor growth and body weight data is sourced from the animal study described in Li et al (Li et al., 2019) with the same vehicle group.

Tumors were measured by length and width in mm twice per week for MDA-MB-435 in SCID mice and by length, width and height in mm once per day for B16F10 in C57BL/6J mice. Tumor volumes were calculated using formula V=(L×W×H×π)/6 (B16F10) or V=L×W×W/2 (MDA-MB-435). If a second tumor was detected in an animal, both tumor volumes were measured and their volumes combined. Animal protocols were approved by the Explora Biolabs' Institutional Animal Care and Use Committee (IACUC) and the UNSW Animal Care and Ethics Committee.

Mass spectrometry. Proteins in serum (50 μl) were precipitated by diluting with acetone (150 μl). The mixture was left for 14 h at 4° C. and protein was pelleted by centrifugation (10 min at 14,000 g) and supernatant removed. The solution (10 μl) was diluted into 40 μl Buffer A (H₂O (0.1% formic acid)) for MS analysis. BT2 standard was prepared by diluting stock suspension of BT2 (10 μg, 30 mg/ml) in Buffer A (990 μl) then diluting 10 μl of this in 990 μl followed by another 1 in 100 dilution. The standard BT2 used for LC mass spectrometry had a concentration of 92 fmol/μl. LC-MS was performed using a Thermo QExactive HF, Gold C₁₈ column (50×2.1 mm) with Solvent A (H₂O (0.1% formic acid) and Solvent B (H₂O:CH₃CN 20:80 (0.1% formic acid) with gradient T=0, 1% B, T=26 min, 100% B, T=27 min, 100%, T=27.1, 1% B, T=30 min, 1% B; column temperature 45° C. MS1 scan, m/z 140-800, 3×10⁶ ions, max IT 25 msec, resolution 120,000 and Top 5 MS2 (2×10⁵ ions, max IT 50 msec, resolution 30,000 and HCD 20, 30 50V). Extracted ion chromatograms (m/z 327.134±5 ppm) were obtained for blanks, standards and samples and integrated peak areas calculated and compared).

RNA-seq and bioinformatics analysis. Jurkat T cells were grown to confluence in 100 mm plates in complete medium and incubated with 10 μM BT2 or vehicle for 4 h. Total RNA was extracted using the RNeasy Mini Kit (Qiagen, cat. no. 74004) with modification. Briefly, cells were washed twice with pre-cooled 1×PBS and TRIzol reagent (Thermo Fisher Scientific cat. no. 15596026) was added to lyse the cells. Chloroform was added to the mixture before microfuge centrifugation at 13000 rpm for 15 min at 4° C. The upper aqueous layer containing total RNA was transferred to fresh microtubes, and isopropanol was added and loaded into RNeasy columns. Columns were washed with buffers RW1 and RPE. Total RNA was eluted from the column using ribonuclease-free water. Samples were submitted to the UNSW Ramaciotti Centre for Genomics for TruSeq Stranded mRNA-seq preparation and sequencing by One NextSeq 500 1×75 bp high-output flow cell with data output up to 400 M reads. Quality control of samples was set at >80% higher than Q30 at 1×75 bp.

RNA-seq reads were first assessed for quality using the tool FastQC (v0.11.8) www.bioinformatics.babraham.ac.uk/projects/fastqc/). The tool Salmon was used for quantifying transcript abundance from RNA-seq reads (Patro et al., 2017). The R package DESeq2 that incorporates a method for differential analysis of count data was then used to identify differentially expressed genes across specific comparisons (Love et al., 2014). This package was also used to perform principal component analysis. Significantly differentially expressed genes were defined as having an adjusted p value <0.05, and a log 2 fold change >1 (absolute fold change >2). The ggplot2 R package was used to prepare bar plots, scatter plots, and density plots. The pheatmap R package was used to generate distance matrices, and clustered heatmaps. DESeq2 output was used to generate a ranked list for input into GSEA Pre-ranked (v6.0.12, Broad Institute) (Subramanian et al., 2005) for pathway analysis against the Curated (Canonical Pathway), Gene Ontology, and Hallmarks gene collections from MSigDb (Liberzon et al., 2015; Liberzon et al., 2011). Ranked scores were calculated by multiplying the sign of the log 2 fold change by the log 10 transformed adjusted p value. Gene sets were only displayed if minimally they had a false discovery rate <0.25, and a maximum of 60 gene sets were displayed in a figure. All immune response related gene sets were selected for display. No other pre-selection was performed on gene sets displayed.

Immunohistochemical staining and analyses. Rabbit monoclonal anti-CD3 (cat. no. ab16669; mouse, rat and human reactive), rabbit monoclonal anti-PD-1 (cat. no. ab214421), rabbit monoclonal anti-CD68 (cat. no. ab125212) antibodies were obtained from Abcam, rabbit monoclonal anti-phospho-ERK (Thr²⁰²/Tyr²⁰⁴) (cat. no. CST4370), rabbit monoclonal total ERK (cat. no. CST4695), antibodies were obtained from Cell Signaling Technology. Rabbit polyclonal anti-PD-L1 (cat. no. PA5-20343) antibody was obtained from ThermoFisher.

Formalin fixed, paraffin embedded sections were prepared from tumors. Heat-induced epitope retrieval was applied to all deparaffinized sections (4 μm sections on Superfrost slides) with citrate buffer, pH 6.0 for 5 min at 110° C. Sections were blocked with Dual Endogenous Enzyme Block (DAKO, S2003) for 10 min and then with 2% skim milk for 20 min. Slides were incubated with primary antibody for 60 min at room temperature or overnight at 4° C. and then for 10 min with probe of MACH3 Rabbit AP-Polymer Detection solution (Biocare Medical, M3R533 G, H, L). After rinsing with buffer, the slides were incubated with polymer of MACH3 Rabbit AP-Polymer Detection solution (Biocare Medical, M3R533 G, H, L) for a further 10 min. Slides were incubated with red chromagen (Warp Red™ Chromagen Kit) for 5 min and counterstained in haematoxylin and Scott blue. Slides were dried with filter paper and dehydrated in xylene then coverslipped. Immunostained slides were scanned using an Aperio ScanScope XT slide scanner (Leica Biosystems, Mt Waverley, Vic, Australia) and images were captured using ImageScope software (Leica Biosystems). Integrated optical density (IOD) or area of positive staining (red chromagen) was assessed using Image-Pro Plus software (Cybernetics, Bethesda, MD, USA). Positive staining was the average of quantitation in 4-8 fields of view under 20× or 40× objectives.

Mouse serum IFN-γ levels. Whole blood was collected from Day 17 mice and spun at 1500 g for 10 min at 4° C. Serum was extracted from the top layer and stored at −80° C. IFN-γ levels were measured using the mouse IFN-γ (Improved) ELISA kit (Invitrogen, cat. no. KMC4021) according to the manufacturer's protocol, with absorbance being read at 450 nm. GraphPad Prism 9 software was used to generate the standard curve. IFN-γ levels in mouse serum were measured alongside the standard curve by using GraphPad Prism 9.

Surface plasmon resonance (SPR). Using a Biacore T200 (Cytiva), CD28 (Sino Biological) was immobilised onto a Series S sensor Chip CM5 (GE) by amine coupling to a level of ˜3000 RU on flow cells 2 and 4 at 25° C., with a flowrate of 10 μl/min. 0.2 M EDC+0.05 M NHS was injected for 420 s followed by CD28 (37.5 μg/ml in 10 mM acetate pH 5) for 450 s with the flowrate reduced to 2 μl/min. Unreacted NHS was blocked with an injection of 1 M ethanolamine-HCl pH 8.5 for 420 s. Flow cells 1 and 3 were used as the reference cells, activated and blocked as described above. Immobilisation running buffer was 20 mM HEPES, 150 mM NaCl, pH 7.5. The SPR run was performed at 12° C. with 20 mM HEPES, 150 mM NaCl, 5% DMSO, pH 7.5 as the running buffer. BT2 was solubilised in DMSO to a concentration of 50 mM, then diluted in running buffer to a final concentration of 20 μM. The concentration was confirmed using ¹H 1D NMR. An 8-point dilution series with 20 μM as the top concentration was performed. Samples were injected for 60 s with a dissociation time of 60 s at a flow rate of 40 μl/min. Solvent correction was performed to correct for excluded volume effects. The integrity of the coupled CD28 was validated by injection of the known binding partner CD80 (Sino Biological) (Waite et al., 2020) (5 point dilution series with 2 μM top concentration).

Systemic pharmacokinetics after oral (PO) and intraperitoneal (IP) administration. Male CD-1 mice were injected with BT2 in Formulation 1, Formulation 2 or Formulation 3 (Table 4) and blood was collected after 0.083, 0.25, 0.5, 1, 2, 4, 8 and 24 h. BT2 was given PO or IP injection.

TABLE 4
Formulation components

| Formulation | Group/Route | Formulation components | BT2 dose |
|---|---|---|---|
| 1 | G1 (PO) | 0.5% (w/v) HPMC, with 0.1% | 30 mg/kg |
| | G2 (IP) | (v/v) Tween 80 in water | |
| 2 | G3 (PO) | 5% (v/v) DMSO in water | 30 mg/kg |
| | G4 (IP) | | |
| 3 | G5 (PO) | 0.01% (v/v) DMSO and 0.5% | 30 mg/kg |
| | G6 (IP) | (v/v) Tween 80 in saline | |

Approximately 110 μl blood samples were collected (n=3/time point, 3 mice/group) into tubes with EDTA-K2 anticoagulant. All blood samples were put on wet ice prior to centrifuging (6000 g, 4° C. for 5 min) to obtain plasma, which were stored at −70° C. or dry ice until analysis.

Plasma samples were analysed by LC-MS/MS (SCIEX ExionLC)-MS/MS (Triple Quad 6500+ With Analyst 1.7.1 AB Sciex). HPLC was performed using a Waters Acquity UPLC HSS T3 1.8 μm, 2.1×50 mm column with Mobile Phase A: H₂O-0.025% FA+1 mM NH₄OAc and Mobile Phase B: MeOH-0.025% FA+1 mM NH₄OAc and injection volume 3 μl.

Statistics. Statistical analysis was performed using Graphpad PRISM v9 noting that PRISM does not draw error bars when these are shorter than the height of the symbol. If distribution was not normal, Mann-Whitney or Kruskal-Wallis was performed as appropriate. Normally distributed data was analyzed by t test or one-way ANOVA as appropriate. Plotted data represent mean±SEM. n indicates biological triplicates rather than technical triplicates. Differences were considered significant when p≤0.05. Where indicated, *p≤0.05, **p<0.01, ***p<0.001, ****p<0.0001.

### Results

### BT2 Inhibits ERK Phosphorylation, Suppresses Migration and Invasion by Human Melanoma Cells and Stimulates Apoptosis

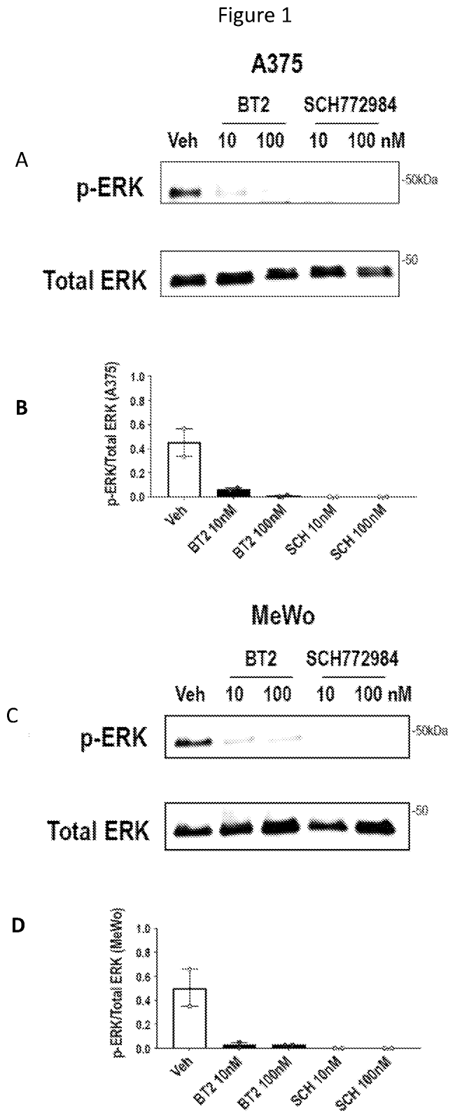

The effect of BT2 on ERK phosphorylation (p-ERK) was evaluated in human melanoma cells given that MAP kinase activation is key to melanoma progression and the paucity of clinically approved ERK inhibitors. Studies by Rossi et al. comparing aggressiveness (combination of growth, invasion and migration rates) among 10 human melanoma cell lines revealed that A375 cells (mutant BRAF^{V600E}) had the most aggressive phenotype (Rossi et al., 2018). MeWo cells (wild type BRAF) are melanoma cells which exhibited among the least aggressive phenotypes studied by Rossi et al. Western blotting was performed with extracts from A375 and MeWo cells incubated with 10 or 100 nM of either BT2 or SCH772984 for 24 h. Membranes were incubated with antibodies to p-ERK (Thr²⁰²/Tyr²⁰⁴) or total ERK followed by secondary antibodies. The results are shown in FIGS. 1A and 1C. Band intensity was quantified using Image J and plotted data represents mean±SEM (FIGS. 1B and 1D). Data represents 2 biologically independent experiments. The results showed that BT2 suppressed ERK phosphorylation as did SCH772984, an ERK inhibitor that overcomes resistance to BRAF and MEK inhibitors and a forerunner of MK-8353 in clinical trials for advanced malignancies.

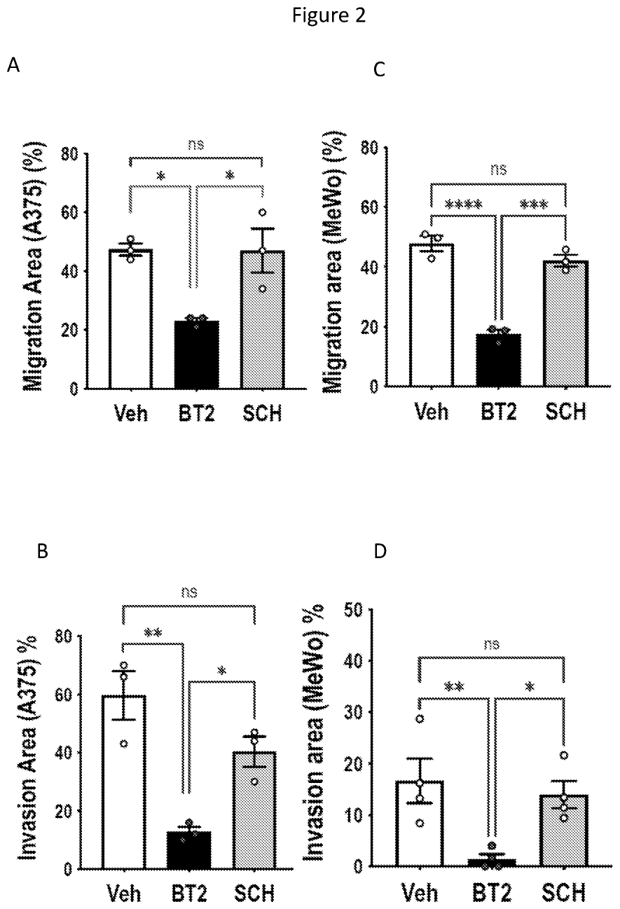

To assess the effect of BT2 on melanoma cell migration, A375 and MeWo cells were incubated with 1 μM of BT2 or SCH772984, and A375 and MeWo migration into the denuded zone was quantified after 24 or 48 h, respectively. The results are shown in FIGS. 2A to 2D. As can be seen, BT2 suppressed melanoma migration (FIGS. 2A and 2C) and invasion (FIGS. 2B and 2D) with greater potency than SCH772984 in both A375 and MeWo cells.

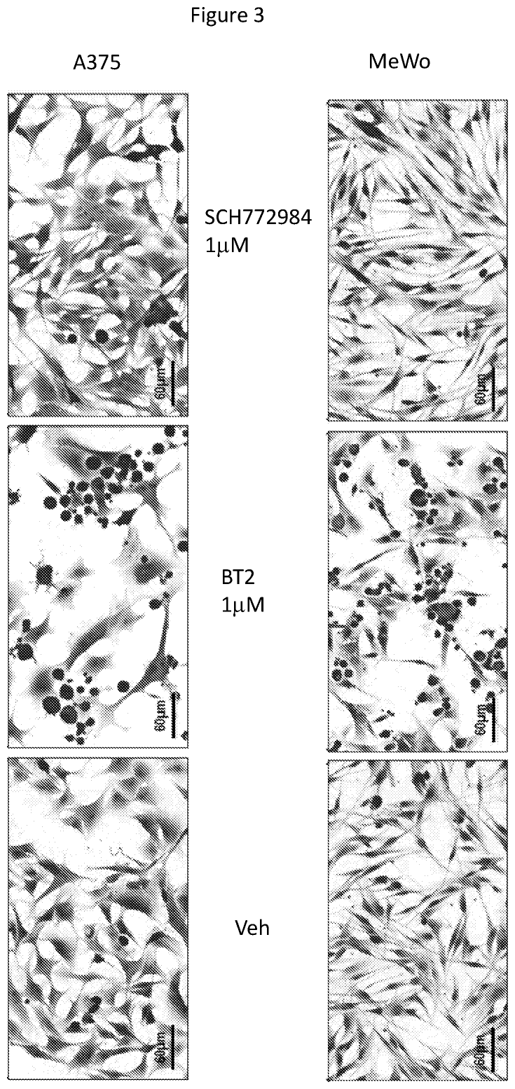

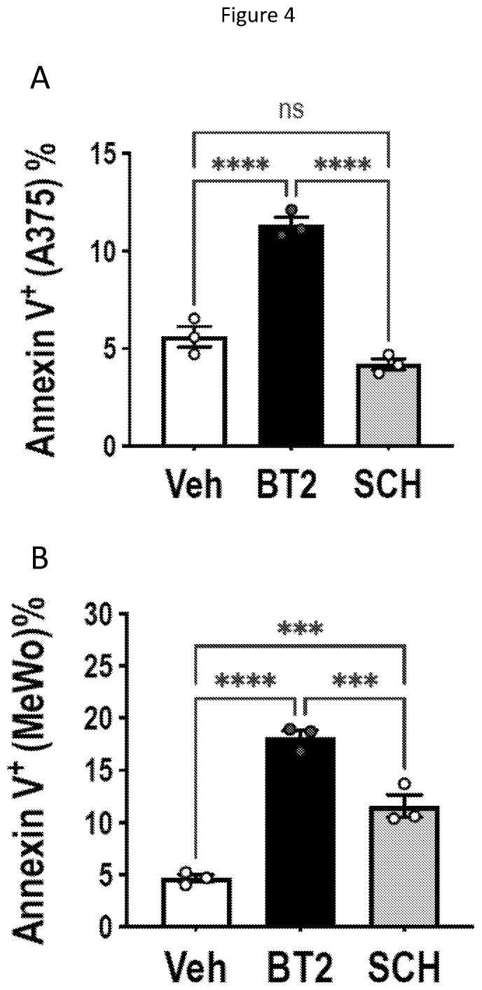

The morphology of A375 and MeWo cells exposed to 1 μM of BT2 or SCH772984 for 24 h was also assessed. The results are shown in FIG. 3. Unlike SCH772984, BT2 had dramatic morphological effects (FIG. 3), causing loss of spindle morphology and cell rounding in both cell lines. Flow cytometry confirmed that BT2 increased apoptosis in both cell lines whereas SCH772984 had no or less potent pro-apoptotic effects (FIG. 4).

BT2 Inhibits Melanoma Growth after Local but not Systemic Delivery in Immunodeficient Mice

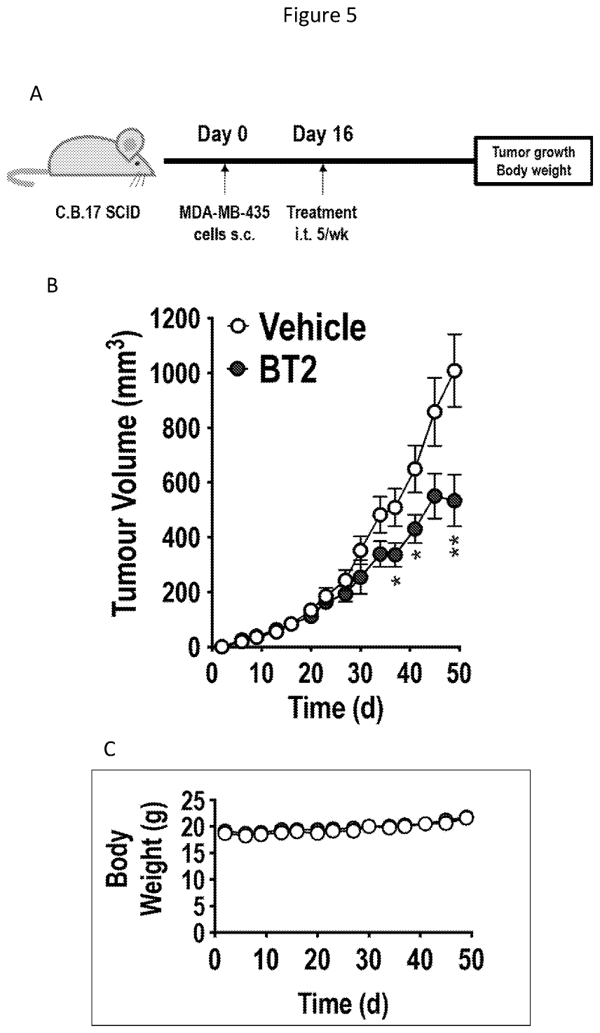

BT2's ability to inhibit melanoma growth was investigated in a murine xenograft model. Immunocompromised SCID mice (C.B.17/lcr-Prkdcscid/lcrlcoCrl) bearing human melanoma (MDA-MB-435 cells, also mutant BRAF^{V600E}) were treated with 20 mg/kg BT2 intratumorally (i.t.) on a 5 days-on/2 days-off regime (FIG. 5A). BT2 caused growth inhibition (FIG. 5B) with no adverse effect on body weight (FIG. 5C).

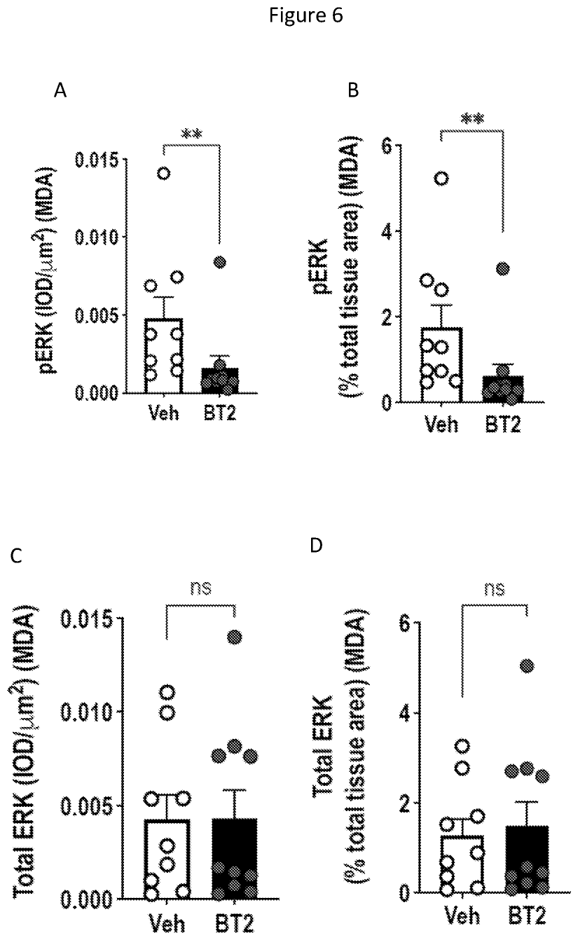

Immunohistochemical staining, using alkaline phosphatase-linked secondary antibodies rather than 3,3′-diaminobenzidine to avoid interference with brown pigment (melanin), confirmed BT2 suppression of ERK phosphorylation in the tumors (FIGS. 6A and 6B) with no effect on total ERK levels (FIGS. 6C and 6D) in this local delivery model.

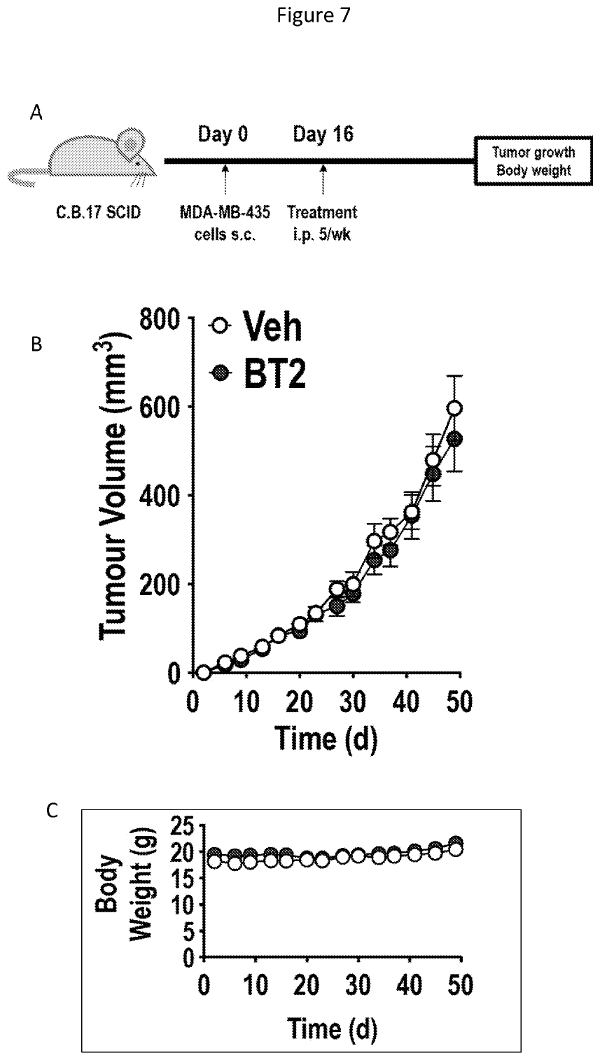

To assess the effect of BT2 when delivered systemically (rather than locally) in the same immunodeficient mouse model, C.B.17 SCID mice bearing s.c. MDA-MB-435 tumors were given BT2 200 mg/kg or vehicle i.p. once a day on a 5 days-on/2 days-off schedule. Treatment commenced on Day 16 (FIG. 7A). The effect on tumor volume and body weight is shown in FIGS. 7B and 7C. Surprisingly, when BT2 was delivered systemically in this model, no inhibition was observed even at 200 mg/kg on a 5 days-on/2 days-off regime (FIG. 7B).

### BT2, Delivered Systemically Inhibits Melanoma Growth and ERK Phosphorylation in a Mouse Immunocompetent Isograft Model Resistant to Tumor Inhibition Using Anti-PD-1 Antibodies

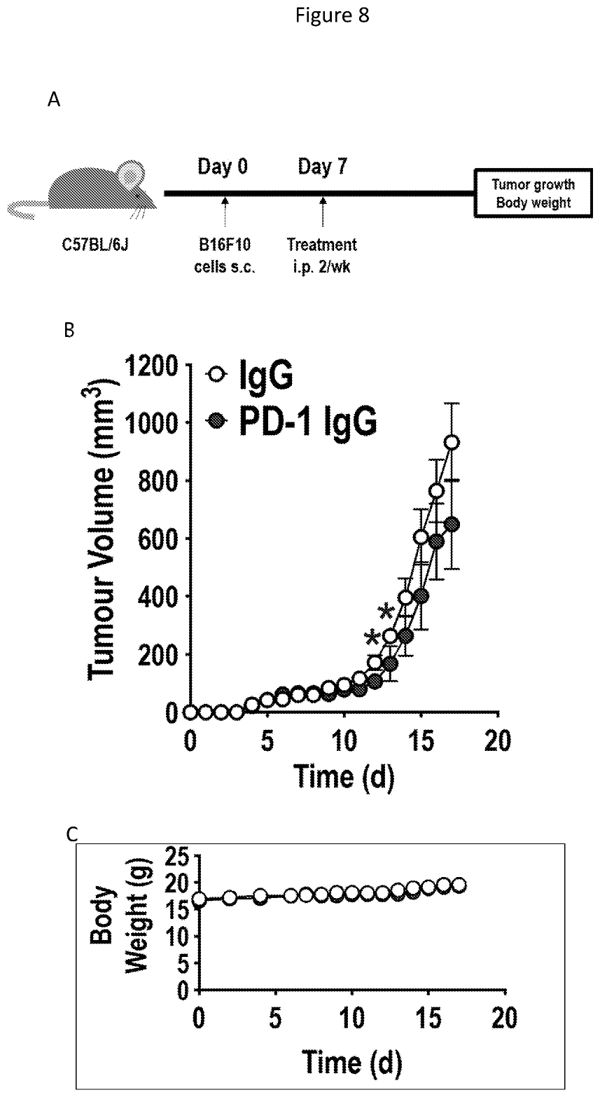

The preceding xenograft studies showing anti-tumor effects in immunocompromised mice when BT2 is delivered intratumorally (i.t.) but not systemically (i.p.) led the inventors to hypothesize that BT2 could rely upon an active immune system in order to effect tumor growth inhibition. Therefore, the effect of BT2 was tested in an immunocompetent mouse model of melanoma growth using B16F10 melanoma. B16F10 grown in C57BL/6 mice provide a highly aggressive, poorly immunogenic, ERK-dependent syngeneic melanoma model. This model is resistant to inhibition with PD-1 antibodies (Kleffel et al., 2015). C57BL/6J mice bearing s.c. B16F10 tumors were administered with an anti-mouse PD-1 monoclonal antibody or control IgG (100 μg, i.p.) twice a week. Treatment commenced on Day 7 (FIG. 8A). Tumor volume and body weight were assessed over time and the results are shown in FIGS. 8B and 8C. These experiments showed that PD-1 antibodies showed transient but unsustained inhibition of B16F10 growth (i.e., Days 12 and 13) (FIG. 8B), as observed by Kleffel et al. (Kleffel et al., 2015).

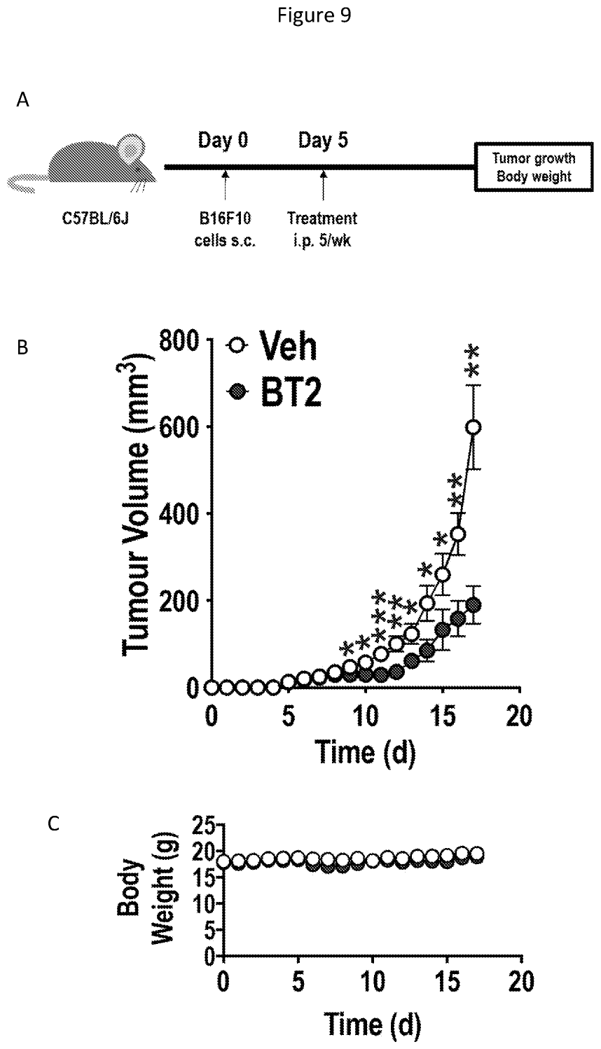

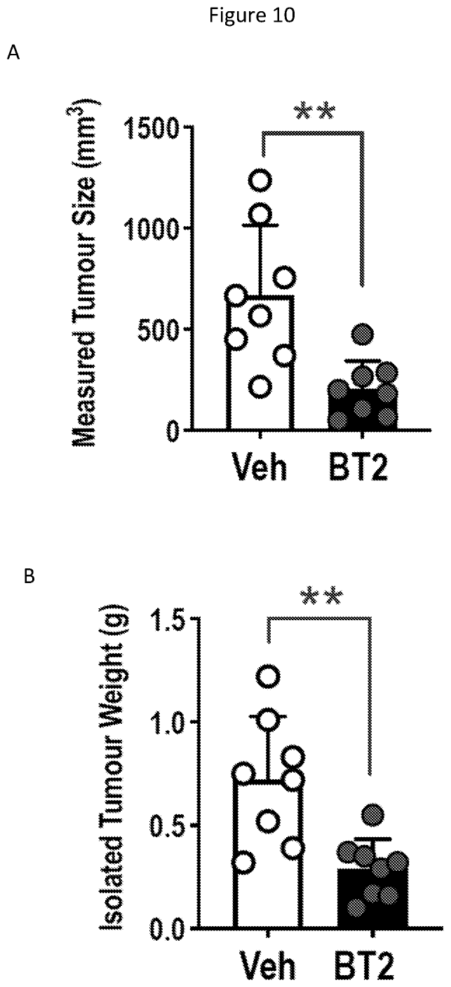

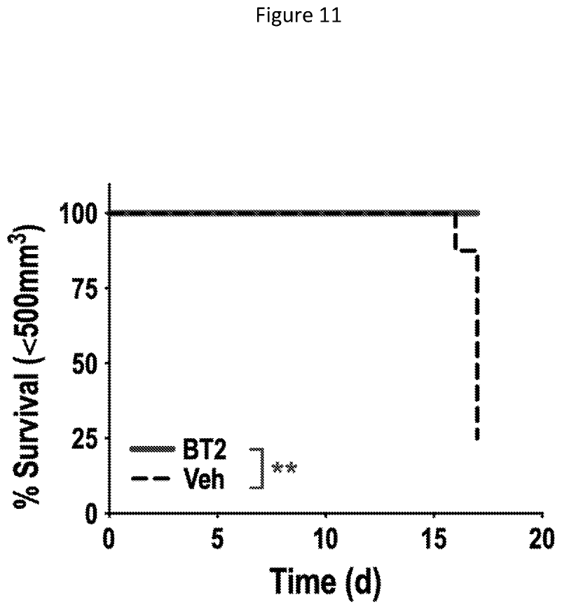

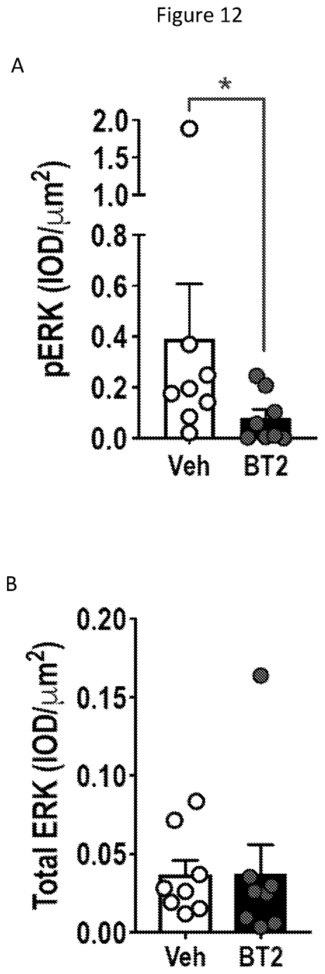

To assess the effect of BT2 in the same model, C57BL/6J mice bearing s.c. B16F10 tumors were administered with BT2 or vehicle (200 mg/kg or 20 ml/kg, respectively, i.p.) once a day on a 5 days-on/2 days-off schedule. Treatment commenced on Day 5 (FIG. 9A). The results of assessment of tumor volume and body weight are shown in FIGS. 9B and 9C, the assessment of tumor size and isolated tumor weight are shown in FIGS. 10A and 10B, and the assessment of survival is shown in FIG. 11. Unlike anti-PD-1 treatment, BT2, delivered systemically, caused significant B16F10 growth inhibition from Day 9, which was sustained for the duration of the study (FIG. 9B) and there was no adverse effect on body weight during this time (FIG. 9C). Tumor size on Day 17 measured using callipers (FIG. 10A) correlated with isolated weighed tumors (FIG. 10B). Kaplan-Meier analysis showed that tumor size in 75% of the animals treated with vehicle exceeded 500 mm³ (Haynes et al., 2018) on Day 17, whereas tumors in none of the BT2 treated mice exceeded this size on Day 17 (FIGS. 10A and 11). LC-MS confirmed the bioavailability of BT2, with serum concentrations of 4 μg/ml or 12.3 μM on Day 17 (data not shown). Immunohistochemical staining of Day 17 tumors revealed BT2 suppression of ERK phosphorylation (FIG. 12A) without affecting total ERK levels (FIG. 12B). These findings, taken together, demonstrate that BT2 inhibits ERK phosphorylation and B16F10 growth in an isograft model resistant to tumor inhibition using anti-PD-1 antibodies.

### BT2 Inhibits Inflammation and Increases Anti-Tumor Immunity in Immunocompetent Mice

Recent studies by the inventors indicate that BT2 possesses anti-inflammatory properties in arthritic mice (Yeh et al., 2021). The inventors hypothesised that this agent may have similar activity within tumors. CD68, a pan-macrophage marker staining in tumor cell nests in melanoma are associated with tumor recurrence and poor survival.

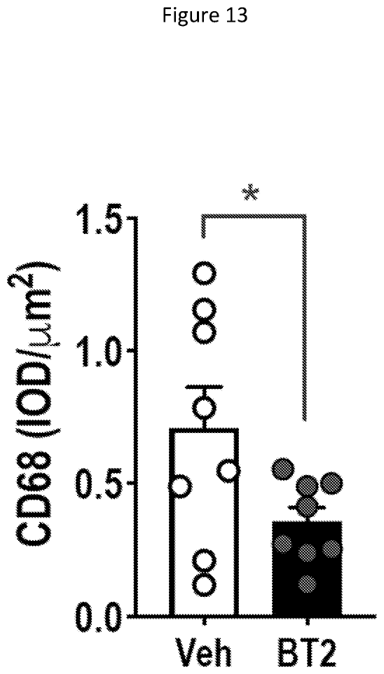

Immunohistochemical analysis was performed on B16F10 tumors (Day 17) from BT2- or vehicle-treated mice with antibodies to CD68. IOD and tissue area were assessed using Image-Pro Plus and the IOD/μm² was determined. The results are shown in FIG. 13. It was found that BT2 reduced levels of CD68 in the tumors (FIG. 13).

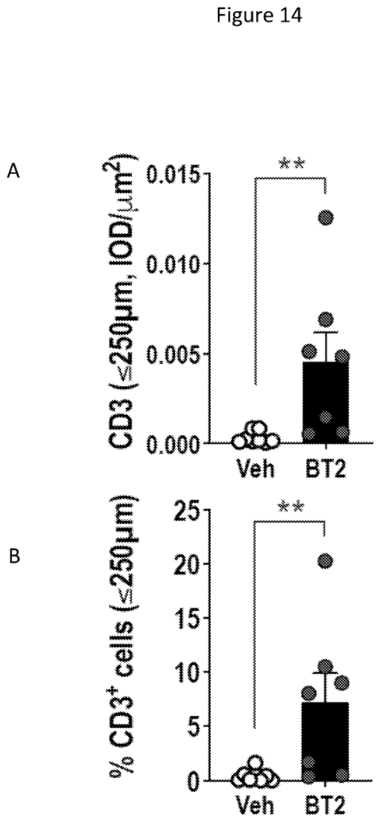

Further immunohistochemical analysis was performed with antibodies to CD3 on B16F10 tumors (Day 17) from BT2- or vehicle-treated mice. Results are shown in FIG. 14. This revealed that BT2 stimulated CD3⁺ staining in the tumor periphery (i.e., within 250 μm) (FIGS. 14A and 14B). B16F10 tumors are immunologically “cold tumors” meaning a general paucity in tumor infiltrating T cells (Bonaventura et al., 2019). Fu et al. found CD3⁺ tumor-infiltrating lymphocytes to be a prognostic marker for overall survival in melanoma (Fu et al., 2019). The inventors' finding of increased peritumoral CD3⁺ staining suggests the involvement of the immune system in the anti-tumor activity of BT2.

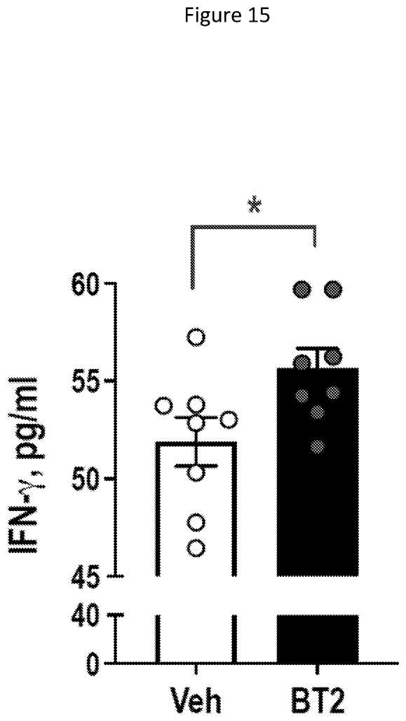

IFN-γ is a biomarker of cellular immunity and the antitumor immune response. Elevated IFN-γ is associated with a systemic immune response in cancer patients and tumor-bearing mice after immune checkpoint therapy. Since IFN-γ is produced by T cells, levels of IFN-γ in the sera of tumor-bearing mice treated with vehicle or BT2 were determined. The results are shown in FIG. 15. The results show that circulating IFN-γ levels were elevated in BT2 treated mice as compared with vehicle-treated mice (FIG. 15).

### BT2 Suppresses PD-1 Expression in Tumors and in Human T Cells

The increased levels of serum IFN-γ in BT2 treated mice encouraged the inventors to determine the effect of this agent on levels of PD-1 within the tumors.

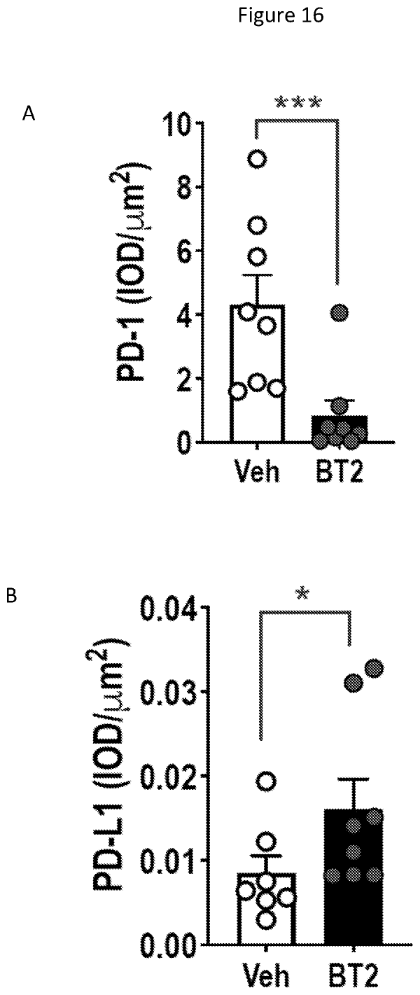

Immunohistochemical analysis was also performed on B16F10 tumors (Day 17) from BT2- or vehicle-treated mice with antibodies to PD-1 and PD-L1. The results are shown in FIGS. 16A and 16B. Immunohistochemical staining revealed that PD-1 levels were reduced in B16F10 tumors treated with BT2 as compared to those treated with vehicle (FIG. 16A). Interestingly, it was found that PD-L1 levels also increased with BT2 treatment (FIG. 16B). These findings have clinical relevance. Studies by Gettinger et al. revealed higher overall response rate (and tendency for greater response) to anti-PD-1 therapy when patients' tumors expressed PD-L1. Similarly, Vilain et al. found that tumor PD-L1 expression was a determinant in patients' response to pembrolizumab/nivolumab. Inspired by these findings, the inventors investigated the effects of BT2 on PD-1 expression on cultured T cells.

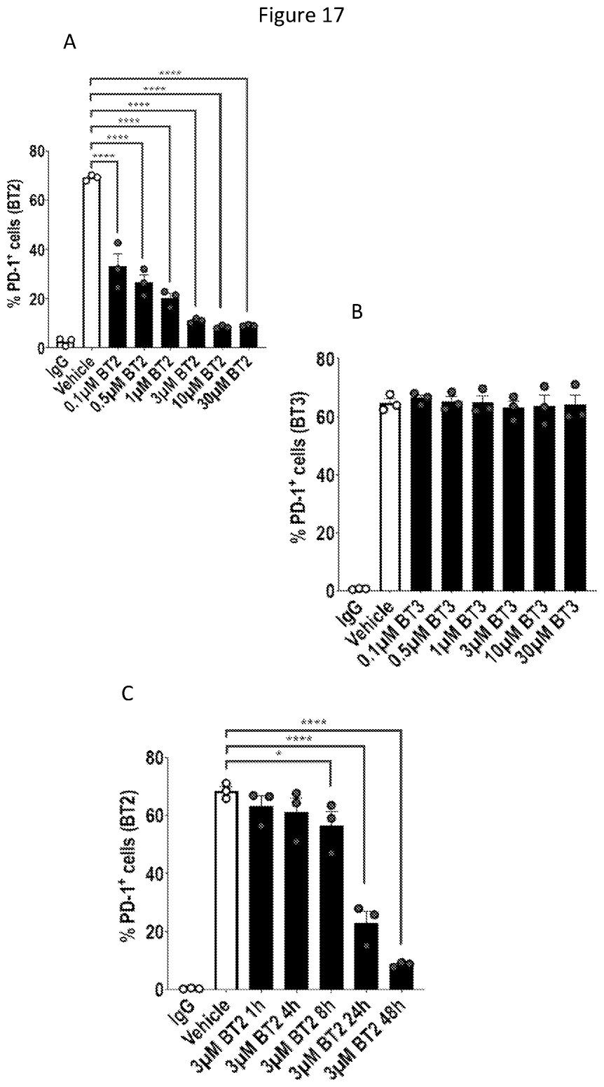

Jurkat T cells express PD-1 (Yi et al., 2023) and are widely used as model human T cells (e.g., Repas et al., 2022). Flow cytometry showed that PD-1 expression in Jurkat T cells was reduced by 50% by 0.1 μM BT2 and 80% by 3 μM (FIG. 17A), concentrations well below those in tumor-bearing mice treated with BT2 in the studies above. BT3 (Li et al., 2020), a structural analogue of BT2 had no inhibitory effect over the same concentration range (FIG. 17B). BT2's effects were not only dose-dependent but time-dependent. BT2 (3 μM) inhibited PD-1 levels by 50% after 24 h and by 80% after 48 h (FIG. 17C).

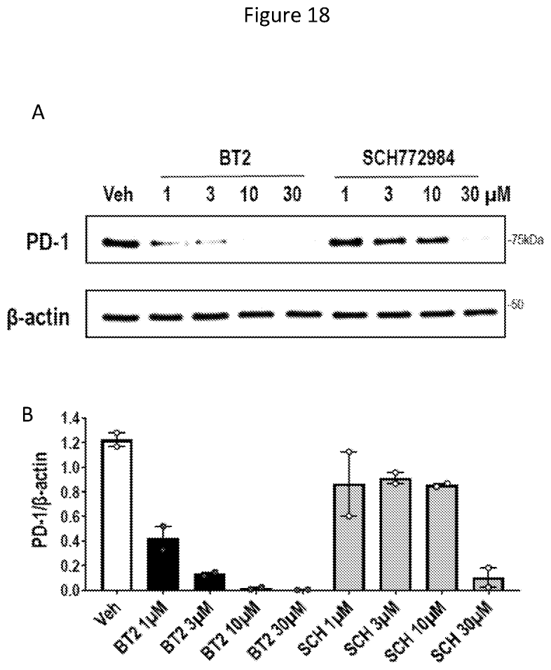

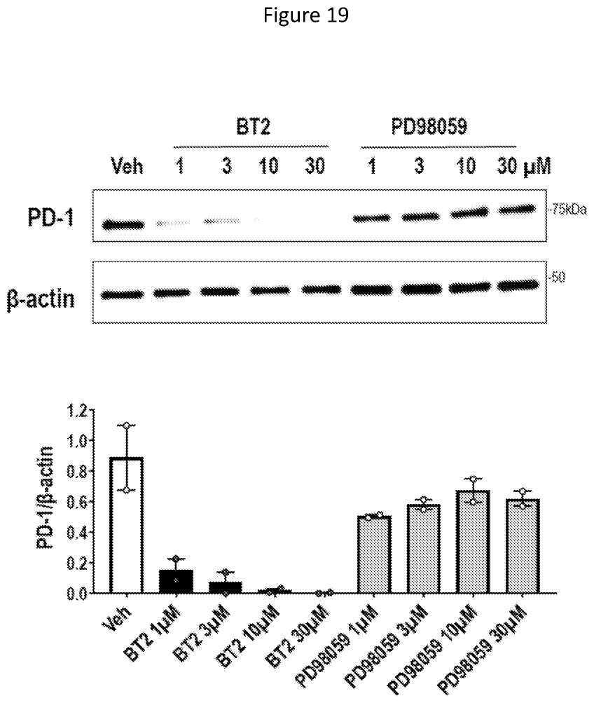

Western blotting confirmed the flow cytometry data by showing that PD-1 expression in Jurkat T cells is suppressed by BT2 in a dose-dependent manner (FIGS. 18A and 18B). SCH772984 also suppressed PD-1 expression but was >10-fold less potent than BT2 (FIGS. 18A and 18B). The MEK1/2 inhibitor PD98059 had no effect on PD-1 expression (FIGS. 19A and 19B).

BT2 Stimulates p-JNK/JUN and Effects Enrichment in Genes Mediating the Immune Response and T Cell Activation

To gain insights into BT2's mode of action we conducted next generation RNA sequencing (RNA-seq) with extracts of Jurkat T cells exposed to 10 μM BT2 for 4 h. Principal component analysis (PCA) and cluster analysis showed clear separation between treatment groups and close association between biological replicates. RNA-seq revealed that from a pool of 19071 gene IDs, there were 856 genes suppressed, and 1320 genes induced by BT2 with an adjusted p value of <0.05, with 14 genes (Table 2), and 30 genes (Table 3) differentially expressed with a ≥2-fold (absolute fold change), respectively.

TABLE 2
Gene expression suppressed at least 2-fold by BT2 in Jurkat T cells

| Gene | | | | BT2 | BT2 | BT2 | log2Fold |
|---|---|---|---|---|---|---|---|
| symbol | Vehicle 1 | Vehicle 2 | Vehicle 3 | Test 1 | Test 2 | Test 3 | Change |
| TUBA1B | 131723.196 | 132368.783 | 127385.704 | 50484.2833 | 49034.778 | 49379.7789 | −1.3939866 |
| TUBA1A | 11763.2926 | 11675.5094 | 11523.4163 | 4997.61651 | 5146.54948 | 5169.36868 | −1.1899578 |
| TUBA1C | 20524.3313 | 20836.3866 | 19428.1989 | 9419.87798 | 9003.87901 | 9492.74933 | −1.1212817 |
| TUBB4B | 17294.8299 | 17350.5585 | 17117.9796 | 6034.02401 | 5953.34497 | 6047.50828 | −1.5200372 |
| TUBB3 | 1565.79087 | 1657.27957 | 1589.47345 | 651.236593 | 705.55867 | 639.012339 | −1.2618223 |
| FAM166A | 805.52781 | 831.158445 | 828.272165 | 312.170933 | 298.545323 | 285.345132 | −1.4427411 |
| TUBB2A | 1288.65974 | 1322.80126 | 1192.37124 | 599.368192 | 644.60997 | 638.007602 | −1.005046 |
| HIST1H2AC | 177.363921 | 191.418309 | 206.535733 | 62.4341866 | 67.1468719 | 71.3362831 | −1.445125 |
| HIST1H2AG | 165.354906 | 162.20183 | 150.111022 | 51.8684012 | 40.2881232 | 74.3504922 | −1.4168756 |
| HIST2H2BE | 133.946712 | 135.000281 | 104.332483 | 35.5394601 | 54.7505263 | 51.2415555 | −1.2734142 |
| TUBA4A | 105.309828 | 123.918168 | 91.5570773 | 33.6184082 | 39.2550944 | 48.2273463 | −1.2528368 |
| MAF | 107.157369 | 125.933098 | 107.526335 | 49.9473493 | 50.6184111 | 48.2273463 | −1.032239 |
| ADAT3 | 51.7311438 | 71.5299995 | 124.56021 | 32.6578822 | 24.7926912 | 20.0947276 | −1.3100481 |
| PRRT1 | 159.812283 | 100.746478 | 72.3939681 | 20.1710449 | 22.7266336 | 60.2841829 | −1.322169 |

TABLE 3
Gene expression increased at least 2-fold by BT2 in Jurkat T cells

| Gene | | | | | | | log2Fold |
|---|---|---|---|---|---|---|---|
| symbol | Vehicle 1 | Vehicle 2 | Vehicle 3 | BT2 | BT2 | BT2 | Change |
| ACTG2 | 598.603235 | 599.441545 | 547.21323 | 3513.60392 | 3448.25013 | 3525.61996 | 2.58075284 |
| JUN | 695.599129 | 698.173094 | 663.256502 | 4079.3537 | 4086.66193 | 4014.92658 | 2.56100848 |
| ENPP1 | 371.355711 | 360.672392 | 391.779122 | 2288.93334 | 2282.99365 | 2243.57634 | 2.59414301 |
| JUND | 3056.75633 | 3222.87984 | 3294.99017 | 7851.3391 | 7882.00973 | 7703.31384 | 1.2901483 |
| GBP1 | 2855.37438 | 2829.96857 | 2617.89364 | 6843.74738 | 6939.88747 | 6948.75681 | 1.31675429 |
| BACH2 | 2766.69242 | 2804.78195 | 2870.20791 | 6174.2608 | 6260.15452 | 6009.3283 | 1.12564875 |
| ZYX | 1087.27779 | 1102.16647 | 1039.06637 | 3059.27515 | 3208.58745 | 3140.80593 | 1.53742792 |
| DUSP8 | 54.502455 | 53.3956334 | 69.2001166 | 826.052316 | 821.257895 | 846.99277 | 3.81081987 |
| FERMT2 | 1431.84416 | 1487.01802 | 1517.07948 | 3161.0909 | 3299.49398 | 3249.31746 | 1.12676589 |
| MYL4 | 206.000805 | 230.709435 | 203.341881 | 896.17071 | 889.437796 | 904.262743 | 2.05207871 |
| PRKCE | 634.630281 | 676.008869 | 650.481096 | 1433.10472 | 1537.14685 | 1488.01458 | 1.17652499 |
| KCNMB1 | 368.584399 | 349.590279 | 406.683762 | 1036.4075 | 994.806733 | 1082.10108 | 1.45546299 |
| CD248 | 54.502455 | 37.2761969 | 50.0370074 | 284.315681 | 291.314121 | 307.449333 | 2.57871482 |
| ATF3 | 475.741769 | 479.553236 | 470.560793 | 975.894364 | 936.957121 | 1002.72691 | 1.01902804 |
| GBP2 | 74.8254044 | 72.5374643 | 93.6863117 | 343.86829 | 332.635273 | 320.510906 | 1.99936262 |
| ARHGAP29 | 211.543427 | 158.171971 | 189.501858 | 550.381368 | 556.802523 | 506.387136 | 1.496712 |
| ACTA2 | 267.893423 | 239.776618 | 319.385153 | 643.552385 | 663.204489 | 658.10233 | 1.2278809 |
| FLNC | 222.628672 | 233.731829 | 211.858818 | 532.131375 | 474.160219 | 510.406082 | 1.15774777 |
| TIMP3 | 58.1975367 | 65.4852108 | 44.7139215 | 202.670975 | 212.803933 | 228.075159 | 1.85039728 |
| IL2RA | 36.0270465 | 35.2612674 | 47.907773 | 168.092041 | 193.176385 | 169.800448 | 2.06007238 |
| ABCA4 | 12.9327859 | 9.06718304 | 10.6461718 | 96.0525948 | 85.7413903 | 106.502056 | 2.99067846 |
| EPAS1 | 172.745069 | 154.142112 | 157.563342 | 332.341978 | 428.706952 | 372.757198 | 1.18697098 |
| SIRPB2 | 30.484424 | 30.2239435 | 26.6154295 | 112.381536 | 120.864369 | 128.606257 | 1.91224247 |
| CREB5 | 58.1975367 | 59.4404221 | 41.5200699 | 179.618352 | 175.614896 | 152.71993 | 1.57733693 |
| NPAS4 | 109.92868 | 110.821126 | 101.138632 | 228.605176 | 253.092056 | 292.378287 | 1.21133144 |
| DMBT1 | 73.9016339 | 52.3881687 | 63.8770307 | 193.065716 | 180.78004 | 166.786239 | 1.4241611 |
| TEAD3 | 115.471303 | 110.821126 | 97.9447804 | 269.907792 | 243.794797 | 226.065686 | 1.13324873 |
| NTN1 | 0 | 1.00746478 | 1.06461718 | 0 | 193.176385 | 146.691512 | 6.50088441 |
| SPECC1L- | 92.3770424 | 125.933098 | 104.332483 | 209.394657 | 316.106812 | 205.970958 | 1.09554415 |
| ADORA2A | | | | | | | |
| ADCYAP1 | 48.9598325 | 40.2985913 | 64.9416479 | 131.592055 | 132.227686 | 130.61573 | 1.24241277 |

Since BT2 was identified from an AP-1-dependent Firefly luciferase screen (Li et al., 2020), it was surprising to discover that among the genes most significantly induced by BT2 in these cells (log FC≥_2) was the proto-oncogene, JUN (Table 3). JUN was induced 5.9-fold by BT2 within 4 h (Table 3). BT2, however, did not induce the expression of all AP-1 family members. For example, while BT2 increased levels of JUND 2.4-fold, it had negligible effects on FOSB and FOS like 2, while FOS was excluded from the analysis because it did not pass the low read filter cut off.

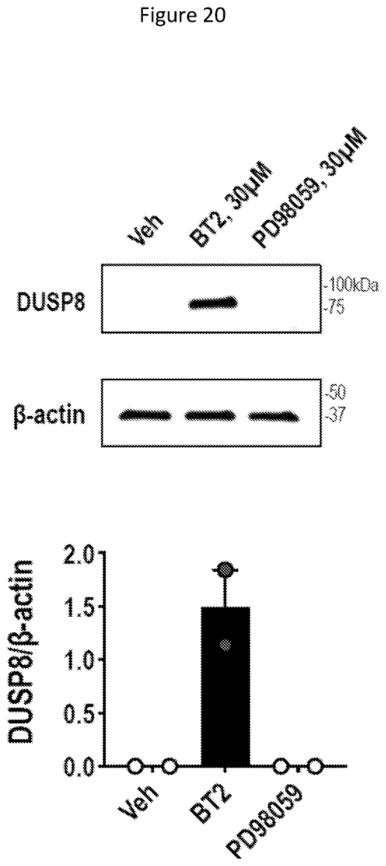

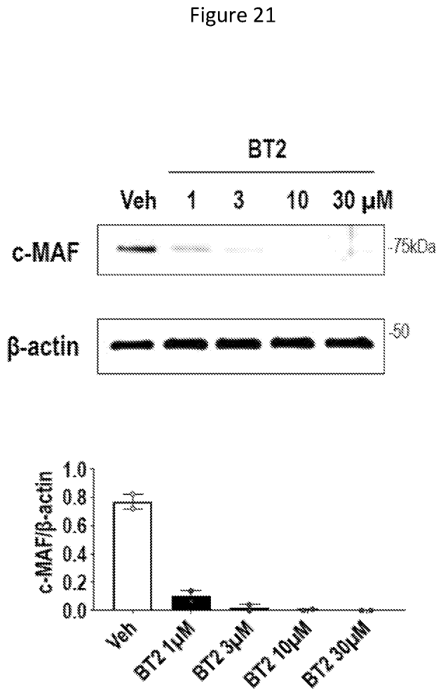

BT2 altered the expression of multiple other genes. For example, BT2 increased the mRNA expression of dual specificity phosphatase 8 (DUSP8) 14.1-fold (Table 3) and inhibited expression of the transcription factor MAF by 2-fold (Table 2). These RNA data were confirmed by Western blotting with extracts from Jurkat T cells incubated with BT2. Blots were incubated with anti-DUSP8 or anti-c-MAF antibodies followed by secondary antibodies. The results are shown in FIGS. 20A and 20B, and 21A and 21B). DUSP8 (FIG. 20) and c-MAF (FIG. 21) protein levels are elevated in Jurkat cells treated with BT2. Gene set enrichment analysis (GSEA) revealed that BT2 effected changes in expression of distinct gene ontologies. Remarkably, there was significant enrichment in genes mediating the immune response and T cell activation, including JUN and multiple other members of the DUSP family.

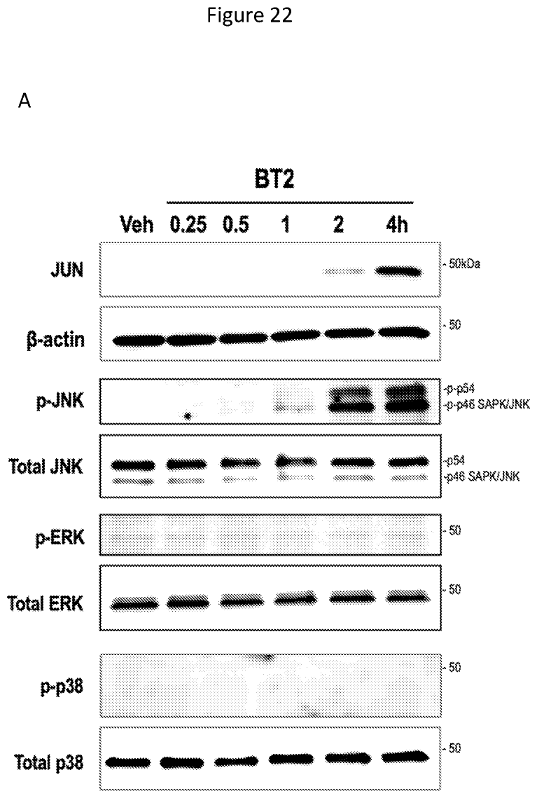

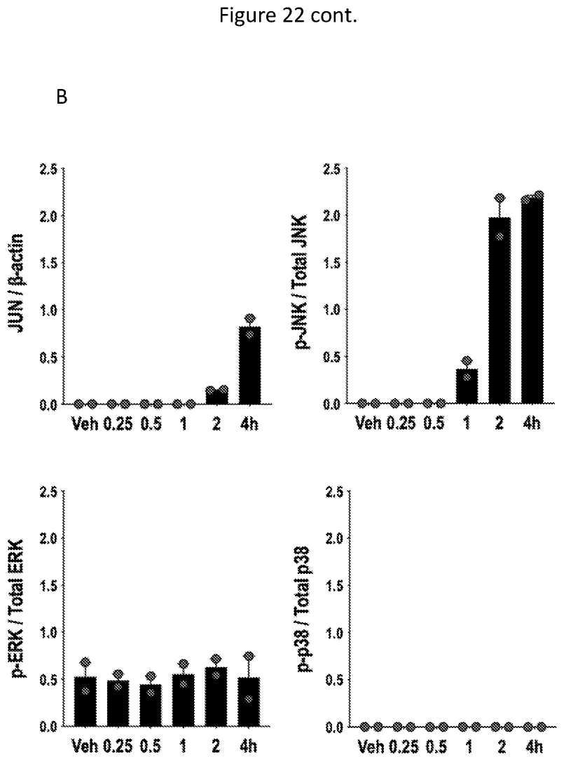

Western blotting was performed with extracts from Jurkat T cells incubated with BT2 for various times, and membranes were incubated with JUN, p-JNK, p-ERK and p38 antibodies followed by secondary antibodies. This confirmed the induction of JUN by BT2 at the protein level within 2-4 h (FIGS. 22A and 22B). BT2 stimulated JNK phosphorylation within 1 h but had no effect on levels of ERK phosphorylation or p38 phosphorylation (FIGS. 22A and 22B).

### BT2 Induces JUN in Tumor Cells Regardless of BRAF Mutation Status

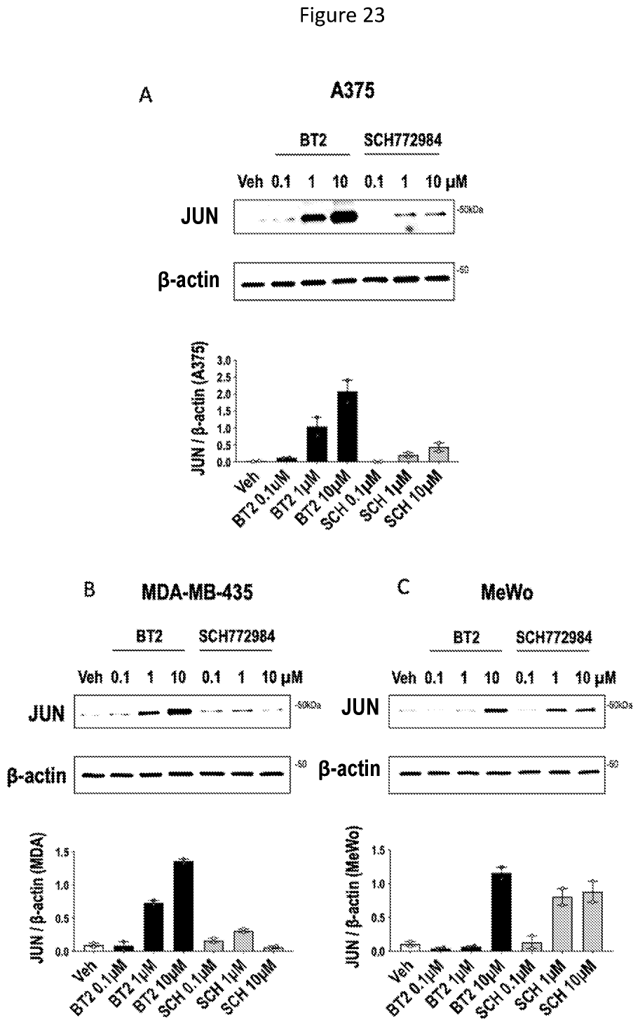

Western blotting was performed to determine the effect of BT2 on JUN expression in multiple melanoma cell lines. A375, MDA-MB-435, MeWo were incubated with various amounts of BT2 or SCH772984 for 24 h prior to preparation of extracts and Western blotting. The results, shown in FIGS. 23A, 23B and 23C, demonstrate that JUN expression was increased by BT2 in all 3 melanoma lines in a dose-dependent manner. The results also demonstrate that JUN was induced by in the tumor cells regardless of BRAF mutation status.

### JUN is Induced by BT2 in T Cells Through CD28 Engagement

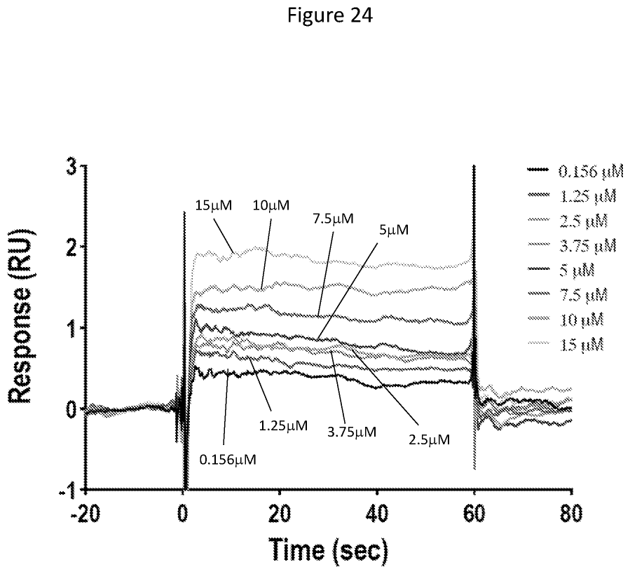

In T cells, the c-jun promoter is activated by anti-CD28 engagement. CD28, a homodimeric cell surface glycoprotein, is a co-stimulatory signal required for T cell activation. The inventors investigated whether BT2 might interact with CD28. In this regard, surface plasmon resonance (SPR) was measure over a concentration range of BT2 from 0.156 to 15 mM using a Biacore T200 at 12° C. in 20 mM HEPES, 150 mM NaCl, 5% DMSO, pH 7.5. The resulting sensorgram is shown in FIG. 24. BT2 bound to CD28 in a dose-dependent manner.

To further assess the interaction between BT2 and CD28, vehicle, BT2 (3 nmol) or a mix of BT2 (3 nmol) and soluble recombinant CD28 (3 nmol) or recombinant epidermal growth factor (3 nmol) was preincubated in growth medium for 30 min at 37° C., then added to Jurkat T cells in 12-well plates (final concentration of BT2, CD28 or EGF was 3 μM). After 24 h, total cell lysates were prepared in RIPA buffer and subjected to Western blotting.

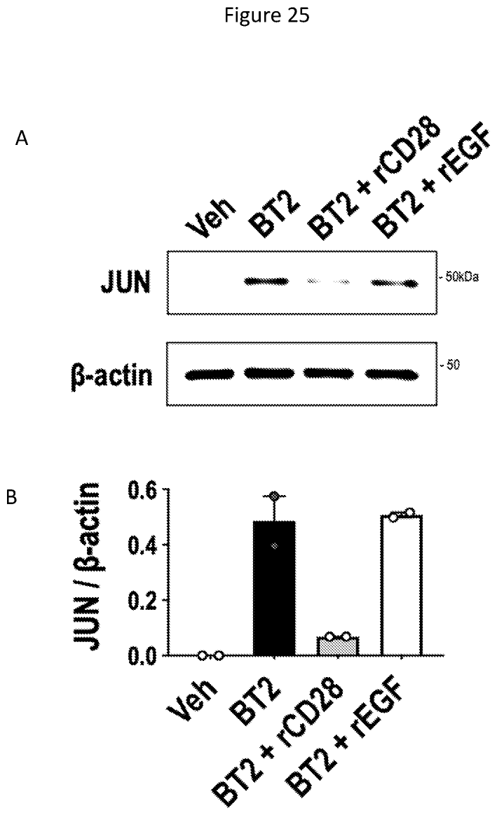

The results are shown in FIGS. 25A and 25B. Preincubation of BT2 with soluble recombinant CD28 inhibited BT2's induction of JUN in the Jurkat T cells, whereas BT2 still induced JUN after preincubation with EGF (FIGS. 25A and 25B).

BT2 is Bioavailable in Circulating Blood after Oral or Intraperitoneal Administration

LC-MS/MS detection was performed for BT2 in plasma of mice given BT2 either by oral gavage (PO) or intraperitoneal (IP) injection. Plasma samples were processed (protein precipitation method used) and injected into LC-MS/MS (SCIEX ExionLC coupled with Triple Quad 6500+).

The highest PO BT2 exposure (AUClast=340 h*ng/mL=1.04 h*nM, Cmax=53.7 ng/ml=0.165 μM at 1 h (Tmax), and 28.8% relative bioavailability) was obtained with Formulation 3 (G5). The next highest PO BT2 exposure (AUClast=189 h*ng/mL=0.579 h*nM, Cmax=57.9 ng/mL=0.177 μM at 15 min (Tmax), and 13.1% relative bioavailability) was obtained with Formulation 1 (G1).

The highest IP BT2 exposure (AUClast=1440 h*ng/mL=4.41 h*nM, Cmax=1073 ng/ml=3.29 μM at 15 min (Tmax), and T1/2=8.72 h) was obtained with Formulation 1 IP (G2). The next highest IP BT2 exposure (AUClast=1182 h*ng/ml=3.62 h*nM, Cmax=846 ng/ml=2.59 μM at 30 min (Tmax), and T1/2=8.42 h) was obtained with Formulation 3 (G6).

### CONCLUSIONS

An immunocompetent mouse model resistant to sustained inhibition by PD-1 antibodies (Kleffel et al., 2015) was used to test the ability of the dibenzoxazepinone BT2 to inhibit PD-1 expression and suppress tumor growth. In tumor cells, BT2 inhibits ERK phosphorylation and increases expression of JUN. In T cells, BT2 increases expression of JUN and reduces expression of PD-1; it also interacts with CD28 and increases phosphorylation of JNK. BT2 inhibited tumor growth in a syngeneic mouse model which is resistant to PD-1 antibody growth inhibition. While B16F10 (BRAF WT) are “cold tumors” (Okada et al., 2020) typifying resistance to immune checkpoint inhibitors in melanoma patients (Bonaventura et al., 2019), Kleffel et al. found that forced overexpression of pdcd1 (which encodes PD-1) enhances B16F10 tumor growth in C57BL/6 mice whereas shRNA knockdown of pdcd1 reduces B16F10 growth (Kleffel et al., 2015). This study revealed that BT2 stimulates JUN in both T cells and melanoma cells and that BT2 negatively regulates PD-1 in T cells. BT2 may be useful in small molecule strategies that increase JUN in T cells to help prevent T cell exhaustion and to maintain effective and sustained tumor cell killing.

BT2 engages CD28 and activates downstream signalling notably JUN, which negatively regulates PD-1 expression and increases T cell activity (Lynn et al., 2019). CD28 is a T cell costimulator, its engagement promoting naive T cell priming. Engagement of CD28 can trigger JUN induction. Anti-CD28 monoclonal antibodies strongly promote T cell activity. CD80- and CD86-Ig RFPs (recombinant fusion protein) targeting CD28 improve T cell responses and inhibit tumor growth. Unlike anti-PD-1 antibodies, BT2 not only inhibits PD-1 expression but engages with CD28 suggesting that T cell activity is enhanced by BT2 and overcome PD-1 antibody resistance. In addition, it was demonstrated herein that BT2 inhibits ERK activation (Thr²⁰²/Tyr²⁰⁴ phosphorylation) in melanoma cells and can stimulate JUN expression in tumor cells regardless of BRAF mutation status (A375 (BRAF^{V600E}), MeWo (BRAF WT), MDA-MB-435 (BRAF^{V600E})) or comparative tumor cell aggression. BT2 suppressed a range of cellular processes including migration and invasion and stimulated tumor cell apoptosis more effectively than the ERK inhibitor SCH772984.

Tumor-associated macrophages (TAM) have been associated clinically with melanoma recurrence and poor survival. TAM density is greater in invasive melanomas as compared with benign melanocytic lesions. Moreover, TAMs promote cancer initiation and progression to malignancy. TAMs create a pro-inflammatory microenvironment and generate IL-1β which is involved in BRAF inhibitor-induced tolerance in melanoma. Numerous chemotherapeutic drugs induce IL-1β processing and production. For example, the BRAF inhibitors dabrafenib and vemurafenib increase IL-1β gene expression and inflammasome activation in dendritic cells and macrophages. BT2 reduced CD68⁺ macrophage accumulation in melanoma.

Besides stimulating JUN, BT2 increased levels of DUSP8 and other members of this family. DUSPs are a group of phosphatases that negatively regulate MAP kinases including JNK, p38 and ERK1/2 and inhibit the production of IL-1β, IL-6 and other key pro-inflammatory cytokines including TNF-α. BT2's induction of DUSPs suggest possible autoregulation involving increased JNK phosphorylation and JUN expression. This has therapeutic implications. For example, DUSP4 regulates responsiveness to MEK inhibition in BRAF wild-type tumors, and its depletion induces resistance to MEK inhibitors. DUSPs are upregulated in T cells stimulated with anti-CD28 and anti-CD3. Recent studies indicate an inverse relationship between DUSP8 and T cell exhaustion. DUSP8 also negatively regulates ERK phosphorylation. BT2 reduced levels of c-MAF, a transcription factor and critical regulator of T cells which is also induced by anti-CD28 and anti-CD3. Increased c-MAF expression in T cells is associated with tumor metastases, its induction requiring IL-6 and TGF-β. c-MAF is a driver of T cell exhaustion, with c-MAF overexpression inhibiting IFN-γ and IL-2 production by T cells and enhancing PD-1 expression, which is associated with T cell dysfunction and exhaustion. Giordano et al. knocked out c-MAF causing increased IFN-γ production in tumor infiltrating lymphocytes (TILs) and, after adoptive transfer, reduced tumor growth and increased survival by tumor-bearing mice. Chihara et al. reported c-MAF deletion inhibits PD-1 expression by T cells. c-MAF functionally interacts with other regulators of T cell function such as PRDM1 which regulate PD-1 expression on T cells. Recently, ChIP-seq of human T cells immunoprecipitated by c-MAF revealed that approximately 70% of induced gene loci associated with c-MAF, and that lentiviral c-MAF overexpression in T cells increased PD-1 expression. By modulating c-MAF and DUSP expression, BT2 may control PD-1 and prevent T cell exhaustion. At the same time, the data herein suggest that BT2 promotes intratumoral T cell infiltration. For example, BT2 increased netrin-1 (NTN1), associated with augmented CD4⁺ T cell chemokinesis and inflammatory cellular infiltration by 90.6-fold.

As a small molecule inhibitor of PD-1, BT2 provides many potential advantages over antibodies or large molecule products which include cheaper production costs, ability to penetrate cell membranes and propensity for oral administration. Moreover, all current approved PD-L1 or PD-1 inhibitors are antibodies requiring intravenous infusion. There is no clinically available small molecule drug able to inhibit both ERK and PD-1. Considering the high cost of immunotherapy, efficacious small molecules could potentially reduce costs to health systems and remove reliance on in-clinic drug administration. Compared to “bi-specific” antibodies, the bifunctional small molecule BT2 is simpler and less expensive to produce. BT2's inhibition of melanoma growth in a model resistant to inhibition by PD-1 antibodies suggests its potential use in patients whose tumors are unresponsive to immune checkpoint immunotherapy. As a potential anti-cancer drug inhibiting both the ERK and PD-1/PD-L1 systems, BT2 could be applied to the treatment of tumors in addition to melanoma, including cutaneous tumors such as, but not confined to, squamous cell carcinoma, basal cell carcinoma, cutaneous sarcoma and Merkel cell carcinoma, and other tumor types such as head and neck cancer, non-small cell lung cancer, urothelial cancer, thyroid cancer, renal cell carcinoma, breast, and hepatocellular carcinoma, particularly where there is evidence of PD-1/PD-L1 inhibition of anti-tumor immune responses.

The capacity of BT2 to serve as both an ERK and PD-1 inhibitor may reduce the risk of treatment-related toxicity, confounding current clinical strategies since one drug, rather than multiple, would be administered. Furthermore, a drug that can inhibit ERK activation and PD-1 function would potentially be an invaluable tool, particularly in circumstances of PD-1 antibody resistance.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

### REFERENCES

Bonaventura, P., et al., 2019. Front Immunol 10, 168.
Chihara, N., et al., 2018. Nature 558, 454-459.
Gettinger, S. et al., 2016. J Clin Oncol 34, 2980-2987.
Haynes, J., et al., 2018. Clin Cancer Res 24, 2116-2127.
Fu, Q., et al. 2019. Oncoimmunology 8, 1593806.
Giordano, M., et al., 2015. EMBO J 34, 2042-2058.
Kleffel, S., et al., 2015. Cell 162, 1242-1256.
Li, Y., et al., 2019. Cancer Lett 459, 268-276.
Li, Y., et al., 2020. Science Advances 6, eaaz7815.
Liberzon, A., et al., 2011. Bioinformatics 27, 1739-1740.
Liberzon, A., et al., 2015. Cell Syst 1, 417-425.
Love, M. I., et al., 2014. Genome Biol 15, 550.
Lynn, R. C., et al., 2019. Nature 576, 293-300.
Neervannan, S. 2006. Expert Opin Drug Metab Toxicol 2, 715-731.
Okada, M., et al., 2020. iScience 23, 101238.
Patro, R., et al., 2017. Nat Methods 14, 417-419.
Repas, J., et al., 2022. Cancers 14, 1343.
Rossi, S., et al., 2018. J Exp Clin Cancer Res 37, 326.
Subramanian, A., et al., 2005. Proc Natl Acad Sci USA 102, 15545-15550.
Waite, J. C., et al., 2020. Sci Transl Med 12, eaba2325.
Vilain, R. et al., 2017. Clin Cancer Res 23, 5024-5033.
Yeh, M. C., et al., 2021. J Inflamm Res 14, 1019-1028.
Yi, B., et al., 2023. Front Immunol 14, 1238694.
Zhang, D., et al., 2022. Front Pharmacol 13, 990505.

1-25. (canceled)

26. A method of increasing immune cell activation and/or treating cancer in a subject in need thereof, comprising administering to the subject an effective amount of a compound of formula (I), or a pharmaceutically acceptable salt thereof: 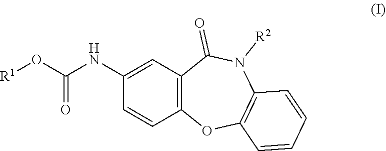
wherein:
R¹ is straight or branched C₁-C₆ alkyl; and
R² is straight or branched C₁-C₆ alkyl, or R² is 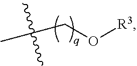
wherein q is 1, 2, 3 or 4; and R³ is straight or branched C₁-C₆ alkyl.

27. The method of claim 26, wherein R¹ is straight C₁-C₆ alkyl or branched C₁-C₆ alkyl.

28. The method of claim 26, wherein R¹ is —CH₂CH₃ or —CH₂CH(CH₃)₂.

29. The method of claim 26, wherein R² is straight C₁-C₆ alkyl or branched C₁-C₆ alkyl.

30. The method of claim 26, wherein R² is —CH₂CH₃ or —CH₂CH(CH₃)₂.

31. The method of claim 26, wherein the compound of formula (I) is a compound of formula (I-1): 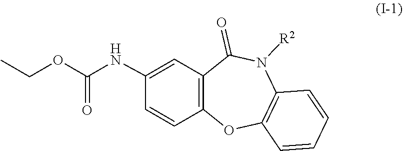
wherein:
R² is straight or branched C₁-C₆ alkyl; or R² is: 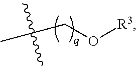
wherein q is 1, 2, 3 or 4; and R³ is straight or branched C₁-C₆ alkyl.

32. The method of claim 26, wherein the compound of formula (I) is a compound of formula (II):

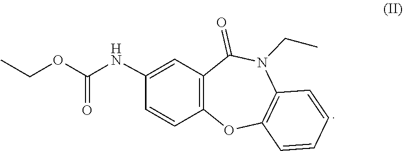 [SMILES: CCOC(=O)Nc1ccc2c(c1)C(=O)N(CC)c1ccccc1O2]

33. The method of claim 26, wherein the compound reduces PD-1 in T cells of the subject.

34. The method of claim 26, wherein the cancer is, or comprises cells that are, resistant to treatment with dabrafenib and trametinib.

35. The method of claim 34, wherein the cancer comprises BRAF mutant or wild type cells.

36. The method of claim 26, wherein the compound increases expression of JUN in tumor cells of the cancer.

37. The method of claim 26, wherein the cancer is resistant to treatment with PD-1 antibody.

38. The method of claim 26, wherein the compound increases DUSP8 expression in T cells of the subject.

39. The method of claim 26, wherein the compound decreases MAF expression is in T cells of the subject.

40. A method of reducing ERK phosphorylation and increasing JUN expression in tumor cells of a subject in need thereof, and/or reducing PD-1 expression, and/or increasing JUN expression, and/or increasing JNK phosphorylation, in T cells of a subject in need thereof, comprising administering an affective amount of a compound of formula (I), or a pharmaceutically acceptable salt thereof: 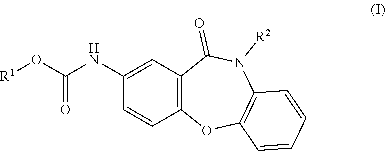
wherein:
R¹ is straight or branched C₁-C₆ alkyl; and
R² is straight or branched C₁-C₆ alkyl, or R² is 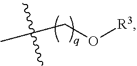
wherein q is 1, 2, 3 or 4; and R³ is straight or branched C₁-C₆ alkyl.

41. The method of claim 40, wherein R¹ is straight C₁-C₆ alkyl or branched C₁-C₆ alkyl.

42. The method of claim 40, wherein R¹ is —CH₂CH₃ or —CH₂CH(CH₃)₂.

43. The method of claim 40, wherein R² is straight C₁-C₆ alkyl or branched C₁-C₆ alkyl.

44. The method of claim 40, wherein R² is —CH₂CH₃ or —CH₂CH(CH₃)₂.

45. The method of claim 40, wherein the compound of formula (I) is a compound of 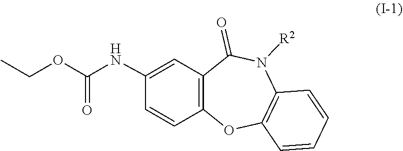
wherein:
R² is straight or branched C₁-C₆ alkyl; or R² is: 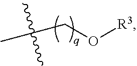
wherein q is 1, 2, 3 or 4; and R³ is straight or branched C₁-C₆ alkyl.

46. The method of claim 40, wherein the compound of formula (I) is a compound of formula (II):

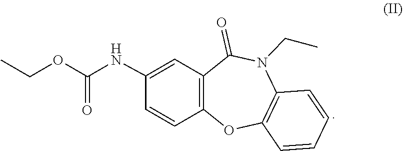 [SMILES: CCOC(=O)Nc1ccc2c(c1)C(=O)N(CC)c1ccccc1O2]

47. The method of claim 40, wherein the compound reduces PD-1 in T cells of the subject.

48. The method of claim 40, wherein the compound increases DUSP8 expression in T cells of the subject.

49. The method of claim 40, wherein the compound decreases MAF expression is in T cells of the subject.

50. The method of claim 40, wherein the tumor cells are resistant to treatment with dabrafenib and trametinib.

51. The method of claim 40, wherein the tumor cells are BRAF mutant or wild type cells.

52. A method of treating a disease or condition associated with PD-1 expression in T cells of a subject, comprising administering an effective amount of a compound of formula (I), or a pharmaceutically acceptable salt thereof formula (I): 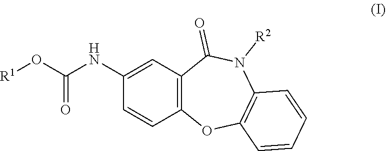
wherein:
R¹ is straight or branched C₁-C₆ alkyl; and
R² is straight or branched C₁-C₆ alkyl, or R² is 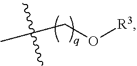
wherein q is 1, 2, 3 or 4; and R³ is straight or branched C₁-C₆ alkyl.

53. The method of claim 52, wherein R¹ is —CH₂CH₃ or —CH₂CH(CH₃)₂, and R² is —CH₂CH₃ or —CH₂CH(CH₃)₂.

54. The method of claim 52, wherein the compound of formula (I) is a compound of formula (II):

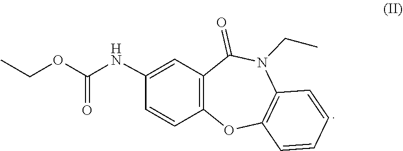 [SMILES: CCOC(=O)Nc1ccc2c(c1)C(=O)N(CC)c1ccccc1O2]

55. The method of claim 52, wherein the disease or condition is cancer in which there is PD-1/PD-L1 inhibition of an anti-tumor immune response.
